(19) United States
(12) Patent Application Publication
(10) Pub. No.: US 2024/0027432 A1
(43) Pub. Date: Jan. 25, 2024

(54) MILD ACID IMMUNOASSAYS FOR DETECTION OF ANALYTES

(71) Applicant: Regeneron Pharmaceuticals, Inc., Tarrytown, NY (US)

(72) Inventors: Joshua Zylstra, South Kent, CT (US); Giane Oliveira Sumner, Mahwah, NJ (US); Michael A. Partridge, Eastchester, NY (US); Jihua Chen, Ossining, NY (US)

(21) Appl. No.: 18/350,867

(22) Filed: Jul. 12, 2023

Related U.S. Application Data
(60) Provisional application No. 63/388,839, filed on Jul. 13, 2022.

(51) Int. Cl. G01N 33/543; G01N 33/53

(52) U.S. Cl. CPC G01N 33/54366; G01N 33/5306

(57) ABSTRACT

The inventions provide immunoassays for reducing the effects of interference in the detection and quantification of analytes, including but not limited to cytokines, enzymes, antibodies, and others by acidifying samples and allowing for binding of the analytes and corresponding capture reagents in the acidified samples without neutralization.

### I. REFERENCE TO RELATED APPLICATIONS

This application claims the benefit of U.S. Provisional Patent Application Ser. No. 63/388,839, filed Jul. 13, 2022, the entire contents of which is incorporated herein by reference.

### II. FIELD OF THE INVENTIONS

The present inventions relate to immunoassays for the detection and quantification of analytes including, but not limited to, cytokines, enzymes, antibodies, and others.

### III. BACKGROUND OF THE INVENTIONS

Immunoassays are a widely used bioanalytical method that measures the presence or concentration of analytes ranging from small molecules to macromolecules in solution based on analyte and antibody interactions.

Immunoassays play an important role in life science research and pharmaceutical analysis, such as diagnosis of disease, therapeutic drug monitoring, and clinical pharmacokinetic and bioequivalence.

The performance of ligand binding assays in complex matrices, such as serum, can be impacted by specific endogenous components interfering with the assay (Zhong, Z. D., et al, AAPS J, 19: 1564 (2017)). Interference in an immunoassay may lead to the misinterpretation of a patient's results by the laboratory and the wrong course of treatment being given by the physician (Tate, J., et al, Clin Biochem Rev. 2004, 25(2): 105-120). For some analytes, the challenges are even greater due to inherent characteristics of the analytes and complicated interactions of endogenous and exogenous components in a biological sample. Commercially available methods are limited by complicated procedures, intolerance to interference, and insensitivity.

Therefore, there is a need in the art for a more sensitive, reliable, and efficient assay and method for the detection and quantification of analytes while avoiding or minimizing interference.

### IV. SUMMARY OF THE INVENTIONS

Acidic buffers, particularly buffer with very low pH, have been employed to dissociate molecules from complexes. For example, affinity tagged antibodies are often eluted from purification column using elution buffer with pH of about 2.0. However, low pH conditions may change conformation of proteins and abrogate their functions, even after the buffers containing the proteins are neutralized. The inventions in this disclosure are based on the surprising findings that mildly acidic conditions can dissociate an analyte of interest from other interfering molecules in a sample, and capture reagents, for example, antibodies, can still bind to the analyte in the mildly acidic conditions, which allows for detection of the analytes in an assay without having to neutralize the sample (in other words, adding a basic solution to the acidified sample to increase the pH of the sample to, for example, neutral pH).

In some aspects, the inventions provide methods for detecting an analyte in a sample comprising the steps of: (i) diluting a sample comprising the analyte with a weak acid to produce an acidified sample, (ii) adding the acidified sample directly to a solid support without neutralizing the acidified sample, wherein the solid support is coated with a capture reagent that can bind to the analyte, (iii) removing the acidified sample from the solid support after a first period of incubation, (iv) adding a detection reagent directly to the solid support, (v) removing the detection reagent from the solid support after a second period of incubation, and (vi) detecting the detection reagent bound to the analyte captured by the capture reagent, wherein the quantity of the detection reagent detected correlates to the quantity of the analyte in the sample. These methods employ a solid support, wherein the solid support is pre-coated with a capture reagent.

The methods can further comprise washing the solid support after the acidified sample is removed from the solid support. The methods can comprise washing the solid support after the detection reagent is removed from the solid support.

In some aspects, the inventions provide methods for detecting an analyte in a sample comprising the steps of: (i) diluting a sample comprising the analyte with a weak acid to produce an acidified sample, (ii) adding a capture reagent to the acidified sample without neutralizing the acidified sample, wherein the capture reagent specifically bind to the analyte, (iii) adding the mixture comparing the acidified sample and the capture reagent to a solid support, wherein the solid support binds specifically to the capture reagent, (iv) removing the mixture of acidified sample and capture reagent from the solid support after a first period of incubation, (vi) adding a detection reagent directly to the solid support, (vii) removing the detection reagent from the solid support after a second period of incubation, and (viii) detecting the detection reagent bound to the analyte captured by the capture reagent, wherein the quantity of the detection reagent detected correlates to the quantity of the analyte in the sample. These methods employ a solid support, wherein the solid support is not pre-coated with a capture reagent and wherein the capture reagent is bound to the solid support after binding with the analyte in the acidified sample.

The analyte can be IL2Rγ, EGFR, an human IgG4 antibody specific for natriuretic peptide receptor 1 (NPR1) (AbA), a human monovalent monoclonal antibody against the anti-NPR1 antibody AbA (AbB), or factor XI.

The sample can be a biological fluid selected from the group consisting of blood, serum, plasma, cerebrospinal fluid (CSF), urine, and saliva.

The sample is from a subject having a disease or disorder. The sample can be from a subject suspected of having a disease or disorder.

The sample can be from a subject that was administered a substance and/or a drug product.

The sample can be diluted at least 3 fold.

The acidified sample can have a pH of about 3.0 to 6.5. The acidified sample can have a pH of about 4.1, about 4.4, or about 4.5.

The weak acid can be an acid that does not completely dissociate into its ions in an aqueous solution selected from the group consisting of acetic acid, citric acid, formic acid, lactic acid, phosphoric acid, and PIPES.

The sample can be acidified for 5 to 120 minutes.

The capture reagent can be an antibody. The capture reagent antibody can be selected from the group consisting of a polyclonal antibody, monoclonal antibody, a bispecific antibody, an Fab fragment, an F(ab′)2 fragment, a monospecific F(ab′)2 fragment, a bispecific F(ab′)2, a trispecific F(ab′)2, a monovalent antibody, an scFv fragment, a diabody, a bispecific diabody, a trispecific diabody, an scFv-Fc, a minibody, an IgNAR, a v-NAR, an hcIgG, and a vhH.

The capture reagent can bind the analyte with a dissociation constant (K_{D}) value of ≤1 μM, ≤100 nM, ≤50 nM, ≤25 nM, ≤20 nM, ≤15 nM, ≤10 nM, ≤5 nM, ≤2 nM, ≤1 nM, ≤0.1 nM, ≤0.01 nM, or ≤0.001 nM.

The methods can further comprise the step of identifying a capture reagent that can bind to the analyte at the pH of the acidified sample.

The capture reagent can be biotinylated and the solid support is coated with streptavidin or avidin.

The capture reagent can bind directly to the solid support.

The detection reagent can be an antibody. The detection reagent antibody can be a polyclonal antibody, monoclonal antibody, a bispecific antibody, an Fab fragment, an F(ab′)2 fragment, a monospecific F(ab′)2 fragment, a bispecific F(ab′)2, a trispecific F(ab′)2, a monovalent antibody, an scFv fragment, a diabody, a bispecific diabody, a trispecific diabody, an scFv-Fc, a minibody, an IgNAR, a v-NAR, an hcIgG, or a vhH.

The detection reagent can be conjugated to a detectable label.

The detectable label can be selected from the group consisting of a rare transition metal particle, a fluorophore, a chromophore, an enzyme, a quantum dot, and noble metal nanoparticles.

The detectable label can be a horseradish peroxidase.

The detectable label can be ruthenium.

The solid support can be an electrochemiluminescence platform.

The sample can comprise an interfering agent that binds to the analyte.

The method can further comprise the step of determining a working pH for the acidified sample at which the interfering agent dissociates partially or completely from the analyte.

The method can recover at least 50%, 55%, 60%, 65%, 70%, 75%, 80%, 85%, 90% or 95% analyte signal in a sample comprising the interfering agent as compared to a sample that does not comprise the interfering agent. The quantity of the analyte can be determined by correlating the amount of detected detection reagent to a predetermined reference standard.

In other aspects, the inventions provide kits for detecting an analyte in a sample, wherein the kit comprises the capture reagent, the detection reagent, and a dilution buffer comprising the weak acid.

In certain aspects, the instant inventions also provide methods for detecting IL2Rγ in a sample comprising the steps of: (i) diluting a sample comprising IL2Rγ with a weak acid to produce an acidified sample, (ii) adding the acidified sample directly to a solid support without neutralizing the acidified sample, wherein the solid support is coated with a capture reagent that can bind to IL2Rγ, (iii) removing the acidified sample from the solid support after a first period of incubation, (iv) adding a detection reagent directly to the solid support, (v) removing the detection reagent from the solid support after a second period of incubation, and (vi) detecting the detection reagent bound to IL2Rγ captured by the capture reagent, wherein the quantity of the detection reagent detected correlates to the quantity of IL2Rγ in the sample.

The methods can further comprise washing the solid support after the acidified sample is removed from the solid support. The methods can comprise washing the solid support after the detection reagent is removed from the solid support.

The sample can be a biological fluid, such as blood, serum, plasma, CSF, urine, or saliva. In some embodiments, the sample is from a subject that was administered an IL2Rγ drug product.

The IL2Rγ drug product can comprise an antibody that binds to IL2Rγ.

The sample can be from a subject diagnosed or suspected of having an IL2Rγ-associated disease or disorder.

IL2Rγ can bind non-covalently to an interfering agent in the sample. The interfering agent can be a human anti-IL2Rγ antibody (for example, Int-IL2Rγ-Ab1). The interfering antibody can be an IL2Rγ-binding fragment thereof.

The acidified sample can have a pH of about 3.4 to 4.5, for example, 4.4 or 4.5.

The weak acid can be an acid that does not completely dissociate into its ions in an aqueous solution including, but not limited to, acetic acid, citric acid, formic acid, lactic acid, phosphoric acid, and PIPES.

The sample can be acidified for 5 to 120 minutes.

The capture reagent can be an IL2Rγ-specific antibody. The capture reagent antibody is a polyclonal antibody, monoclonal antibody, a bispecific antibody, an Fab fragment, an F(ab′)2 fragment, a monospecific F(ab′)2 fragment, a bispecific F(ab′)2, a trispecific F(ab′)2, a monovalent antibody, an scFv fragment, a diabody, a bispecific diabody, a trispecific diabody, an scFv-Fc, a minibody, an IgNAR, a v-NAR, an hcIgG, or a vhH. The capture reagent is biotinylated, and the solid support is coated with streptavidin. The capture reagent can bind directly to the solid support.

The detection reagent can be an antibody. The detection reagent antibody can be a polyclonal antibody, monoclonal antibody, a bispecific antibody, an Fab fragment, an F(ab′)2 fragment, a monospecific F(ab′)2 fragment, a bispecific F(ab′)2, a trispecific F(ab′)2, a monovalent antibody, an scFv fragment, a diabody, a bispecific diabody, a trispecific diabody, an scFv-Fc, a minibody, an IgNAR, a v-NAR, an hcIgG, or a vhH.

The detection reagent can be conjugated to a detectable label. The detectable label can be selected from the group consisting of a rare transition metal particle, a fluorophore, a chromophore, an enzyme, a quantum dot, and noble metal nanoparticles.

The detectable label can be a horseradish peroxidase. The detectable label can be ruthenium.

The solid support can be an electrochemiluminescence platform.

The capture reagent and/or the detection reagent can be selected from human anti-IL2Rγ monoclonal antibodies (anti-IL2Rγ-Ab2 and anti-IL2Rγ-Ab1).

The method can recover at least 50%, 55%, 60%, 65%, 70%, 75%, 80%, 85%, 90%, 95%, or 100% analyte signal in a sample comprising the interfering agent compared to a sample that does not comprise the interfering agent. The method can recover at least 70%-100% analyte signal in a sample comprising the interfering agent as compared to a sample that does not comprise the interfering agent.

The quantity of the analyte can be determined by correlating the amount of detected detection reagent to a predetermined reference standard.

In other aspects, the inventions provides kits for detecting IL2Rγ in a sample, wherein the kit comprises the capture reagent, the detection reagent, and a dilution buffer comprising the weak acid.

In some aspects, the methods for detecting IL2Rγ in a sample comprise the steps of: (i) diluting a sample comprising IL2Rγ with a weak acid to produce an acidified sample, (ii) adding the acidified sample directly to a solid support without neutralizing the acidified sample, wherein the solid support is coated with a capture reagent, wherein the capture reagent is a first IL2Rγ-specific antibody, (iii) removing the acidified sample from the solid support after a first period of incubation, (iv) adding a detection reagent directly to the solid support, wherein the detection reagent is a second IL2Rγ-specific antibody (v) removing the detection reagent from the solid support after a second period of incubation, and (vi) detecting the detection reagent bound to IL2Rγ captured by the capture reagent, wherein the quantity of the detection reagent detected correlates to the quantity of IL2Rγ in the sample.

The capture reagent and/or the detection reagent in the kit can be selected from human anti-IL2Rγ monoclonal antibodies (anti-IL2Rγ-Ab2 and anti-IL2Rγ-Ab1).

The acidified sample can have a pH of about 3.4 to 4.5, for example, 4.4 or 4.5.

The assay can have an analyte recovery rate of about at least 50%, 55%, 60%, 65%, 70%, 75%, 80%, 85%, 90%, 95%, or 100% in the presence of 100-1000 μg/mL interfering agent (for example, human monoclonal anti-IL2Rγ antibody, Int-IL2Rγ-Ab1).

In certain aspects, the inventions provide methods for detecting EGFR in a sample comprising the steps of: (i) diluting a sample comprising EGFR with a weak acid to produce an acidified sample, (ii) adding the acidified sample directly to a solid support without neutralizing the acidified sample, wherein the solid support is coated with a capture reagent that can bind to EGFR, (iii) removing the acidified sample from the solid support after a first period of incubation, (iv) adding a detection reagent directly to the solid support, (v) removing the detection reagent from the solid support after a second period of incubation, and (vi) detecting the detection reagent bound to EGFR captured by the capture reagent, wherein the quantity of the detection reagent detected correlates to the quantity of EGFR in the sample.

The methods can further comprise washing the solid support after the acidified sample is removed from the solid support. The methods can comprise washing the solid support after the detection reagent is removed from the solid support.

The sample can be a biological fluid, such as blood, serum, plasma, CSF, urine, or saliva.

The sample can be from a subject that was administered an EGFR drug product.

The EGFR drug product can comprise an antibody that binds to EGFR.

The sample can be from a subject diagnosed or suspected of having an EGFR-associated disease or disorder.

EGFR can bind non-covalently to an interfering agent in the sample, wherein the interfering agent is an antibody.

The interfering antibody can be Int-EGFR-Ab1 or an EGFR-binding fragment thereof.

The acidified sample can have a pH of about 4.0-6.0, for example, 4.5 or 5.0.

The weak acid can be an acid that does not completely dissociate into its ions in an aqueous solution including, but not limited to, acetic acid, citric acid, formic acid, lactic acid, phosphoric acid, and PIPES.

The sample can be acidified for 5 to 120 minutes.

The capture reagent can be an EGFR-specific antibody. The capture reagent antibody can be a polyclonal antibody, monoclonal antibody, a bispecific antibody, an Fab fragment, an F(ab′)2 fragment, a monospecific F(ab′)2 fragment, a bispecific F(ab′)2, a trispecific F(ab′)2, a monovalent antibody, an scFv fragment, a diabody, a bispecific diabody, a trispecific diabody, an scFv-Fc, a minibody, an IgNAR, a v-NAR, an hcIgG, or a vhH.

The capture reagent can be biotinylated and the solid support is coated with streptavidin. The capture reagent can bind directly to the solid support.

The detection reagent can be an antibody. The detection reagent antibody can be a polyclonal antibody, monoclonal antibody, a bispecific antibody, an Fab fragment, an F(ab′)2 fragment, a monospecific F(ab′)2 fragment, a bispecific F(ab′)2, a trispecific F(ab′)2, a monovalent antibody, an scFv fragment, a diabody, a bispecific diabody, a trispecific diabody, an scFv-Fc, a minibody, an IgNAR, a v-NAR, an hcIgG, or a vhH.

The detection reagent can be conjugated to a detectable label. The detectable label can be selected from the group consisting of a rare transition metal particle, a fluorophore, a chromophore, an enzyme, a quantum dot, and noble metal nanoparticles.

The detectable label can be a horseradish peroxidase. The detectable label can be ruthenium.

The solid support can be an electrochemiluminescence platform.

The capture reagent and/or the detection reagent can be selected from the EGFR antibodies disclosed in WO 2014/004427 and the EGFR antibodies in R&D Systems Human EGFR Quantikine Kit DEGFR0.

The method can recover at least 50%, 55% 60%, 65%, 70%, 75%, 80%, 85%, 90%, 95% or 100% analyte signal in a sample comprising the interfering agent compared to a sample that does not comprise the interfering agent.

The quantity of the analyte can be determined by correlating the amount of detected detection reagent to a predetermined reference standard.

In other aspects, the inventions provide kits for detecting EGFR in a sample, wherein the kits comprise the capture reagent, the detection reagent, and a dilution buffer comprising the weak acid.

In some aspects, the methods for detecting EGFR in a sample comprise the steps of: (i) diluting a sample comprising EGFR with a weak acid to produce an acidified sample, (ii) adding the acidified sample directly to a solid support without neutralizing the acidified sample, wherein the solid support is coated with a capture reagent, wherein the capture reagent is a first EGFR-specific antibody, (iii) removing the acidified sample from the solid support after a first period of incubation, (iv) adding a detection reagent directly to the solid support, wherein the detection reagent is a second EGFR-specific antibody (v) removing the detection reagent from the solid support after a second period of incubation, and (vi) detecting the detection reagent bound to EGFR captured by the capture reagent, wherein the quantity of the detection reagent detected correlates to the quantity of EGFR in the sample.

The capture reagent and/or the detection reagent can be selected from the EGFR antibodies disclosed in WO 2014/004427 and the EGFR antibodies in R&D Systems Human EGFR Quantikine Kit DEGFR0.

The acidified sample can have a pH of about 4.0-6.0, for example, 4.5 or 5.0.

The assay can have analyte recovery rate of at least 55%, 60%, 65%, 70%, 75%, 80%, 85%, 90%, 95%, or 100% in the presence of at least 0-2000 μg/mL interfering agent (for example, Int-EGFR-Ab1).

The assay can detect EGFR at a concentration of as low as 0.31 ng/mL in the presence of 2% human serum, or 15.6 ng/mL in undiluted human serum.

In certain aspects, the instant inventions provides methods for detecting AbA, a human monoclonal antibody of IgG4 subclass specific for Natriuretic Peptide Receptor 1, in a sample comprising the steps of: (i) diluting a sample comprising AbA with a weak acid to produce an acidified sample, (ii) adding the acidified sample directly to a solid support without neutralizing the acidified sample, wherein the solid support is coated with a capture reagent that can bind to AbA, (iii) removing the acidified sample from the solid support after a first period of incubation, (iv) adding a detection reagent directly to the solid support, (v) removing the detection reagent from the solid support after a second period of incubation, and (vi) detecting the detection reagent bound to AbA captured by the capture reagent, wherein the quantity of the detection reagent detected correlates to the quantity of AbA in the sample.

The methods can further comprise washing the solid support after the acidified sample is removed from the solid support. The methods can comprise washing the solid support after the detection reagent is removed from the solid support.

The sample can be a biological fluid such as blood, serum, plasma, CSF, urine, or saliva.

The sample can be from a subject diagnosed or suspected of having an natriuretic peptide receptor 1-associated disease or disorder.

The sample can be from a subject that was administered AbA.

The AbA can bind non-covalently to an interfering agent in the sample.

The interfering agent can be an antibody. The interfering antibody can include one or more AbA reversal agents that are human anti-AbA monoclonal antibodies (Int-AbA-Ab1, Int-AbA-Ab2, AbB, Int-AbA-Ab3), or an antigen binding fragment thereof.

The acidified sample can have a pH of about 4.0-6.0, for example, about 4.5 or 5.0.

The weak acid can be an acid that does not completely dissociate into its ions in an aqueous solution including, but not limited to, acetic acid, citric acid, formic acid, lactic acid, phosphoric acid, and PIPES.

The sample can be acidified for 5 to 120 minutes.

The capture reagent can be a AbA-specific antibody. The capture reagent antibody can be a polyclonal antibody, monoclonal antibody, a bispecific antibody, an Fab fragment, an F(ab′)2 fragment, a monospecific F(ab′)2 fragment, a bispecific F(ab′)2, a trispecific F(ab′)2, a monovalent antibody, an scFv fragment, a diabody, a bispecific diabody, a trispecific diabody, an scFv-Fc, a minibody, an IgNAR, a v-NAR, an hcIgG, or a vhH.

The capture reagent can be biotinylated and the solid support is coated with avidin or streptavidin. The capture reagent can bind directly to the solid support.

The detection reagent can be an antibody. The detection reagent antibody can be a polyclonal antibody, monoclonal antibody, a bispecific antibody, an Fab fragment, an F(ab′)2 fragment, a monospecific F(ab′)2 fragment, a bispecific F(ab′)2, a trispecific F(ab′)2, a monovalent antibody, an scFv fragment, a diabody, a bispecific diabody, a trispecific diabody, an scFv-Fc, a minibody, an IgNAR, a v-NAR, an hcIgG, or a vhH.

The detection reagent can be conjugated to a detectable label.

The detectable label can be selected from the group consisting of a rare transition metal particle, a fluorophore, a chromophore, an enzyme, a quantum dot, and noble metal nanoparticles.

The detectable label can be a horseradish peroxidase. The detectable label can be ruthenium.

The solid support can be an electrochemiluminescence platform.

The capture reagent can be a mouse anti-AbA monoclonal antibody (anti-AbA-Ab1) and/or the detection reagent is selected from a mouse anti-AbA antibody (anti-AbA-Ab2) and a mouse anti-human IgG4 Fc specific monoclonal antibody (anti-hIgG4Fc).

The method can recover at least 50%, 55%, 60%, 65%, 75%, 80%, 85%, 90%, 95% or 100% analyte signal in a sample comprising an interfering agent compared to a sample that does not comprise the interfering agent.

The quantity of the analyte can be determined by correlating the amount of detected detection reagent to a predetermined reference standard.

In other aspects, the present inventions provide kits for detecting AbA in a sample, wherein the kits comprise the capture reagent, the detection reagent, and a dilution buffer comprising the weak acid.

In some aspects, the methods for detecting AbA in a sample comprise the steps of: (i) diluting a sample comprising AbA with a weak acid to produce an acidified sample, (ii) adding the acidified sample directly to a solid support without neutralizing the acidified sample, wherein the solid support is coated with a capture reagent, wherein the capture reagent is a first AbA-specific antibody, (iii) removing the acidified sample from the solid support after a first period of incubation, (iv) adding a detection reagent directly to the solid support, wherein the detection reagent is a second AbA-specific antibody (v) removing the detection reagent from the solid support after a second period of incubation, and (vi) detecting the detection reagent bound to AbA captured by the capture reagent, wherein the quantity of the detection reagent detected correlates to the quantity of AbA in the sample.

The assay can have an analyte recovery rate of about at least 50%, 55%, 60%, 65%, 75%, 80%, 85%, 90%, 95%, or 100% in the presence of at least 0-2 mg/mL interfering agent (for example, human monoclonal antibody against AbA).

The assay can detect AbA at a concentration of as low as 0.27 ng/mL in the presence of 2% human serum, or 13.7 ng/mL in undiluted human serum.

In certain aspects, the instant inventions provide methods for detecting AbB, a human monoclonal antibody against AbA, in a sample comprising the steps of: (i) diluting a sample comprising AbB with a weak acid to produce an acidified sample, (ii) adding the acidified sample directly to a solid support without neutralizing the acidified sample, wherein the solid support is coated with a capture reagent that can bind to AbB, (iii) removing the acidified sample from the solid support after a first period of incubation, (iv) adding a detection reagent directly to the solid support, (v) removing the detection reagent from the solid support after a second period of incubation, and (vi) detecting the detection reagent bound to AbB captured by the capture reagent, wherein the quantity of the detection reagent detected correlates to the quantity of AbB in the sample.

The methods can further comprise washing the solid support after the acidified sample is removed from the solid support. The methods can comprise washing the solid support after the detection reagent is removed from the solid support.

The sample can be a biological fluid, such as blood, serum, plasma, CSF, urine, or saliva.

The sample can be from a subject that was administered a AbB drug product.

The AbB drug product can comprise an antibody that binds to AbB.

The sample can be from a subject diagnosed or suspected of having a disease or disorder.

The AbB can bind non-covalently to an interfering agent in the sample.

The interfering agent can be an antibody.

The interfering antibody can be AbA or a AbB-binding fragment thereof.

The acidified sample can have a pH of about 4.0-6.0, for example, about 4.5 or 5.0.

The weak acid can be an acid that does not completely dissociate into its ions in an aqueous solution including, but not limited to, acetic acid, citric acid, formic acid, lactic acid, phosphoric acid, and PIPES.

The sample can be acidified for 5 to 120 minutes.

The capture reagent can be a AbB-specific antibody.

The capture reagent antibody can be a polyclonal antibody, monoclonal antibody, a bispecific antibody, an Fab fragment, an F(ab′)2 fragment, a monospecific F(ab′)2 fragment, a bispecific F(ab′)2, a trispecific F(ab′)2, a monovalent antibody, an scFv fragment, a diabody, a bispecific diabody, a trispecific diabody, an scFv-Fc, a minibody, an IgNAR, a v-NAR, an hcIgG, or a vhH.

The capture reagent can be biotinylated and the solid support is coated with streptavidin.

The capture reagent can bind directly to the solid support.

The detection reagent can be an antibody.

The detection reagent antibody can be a polyclonal antibody, monoclonal antibody, a bispecific antibody, an Fab fragment, an F(ab′)2 fragment, a monospecific F(ab′)2 fragment, a bispecific F(ab′)2, a trispecific F(ab′)2, a monovalent antibody, an scFv fragment, a diabody, a bispecific diabody, a trispecific diabody, an scFv-Fc, a minibody, an IgNAR, a v-NAR, an hcIgG, or a vhH.

The detection reagent can be conjugated to a detectable label.

The detectable label can be selected from the group consisting of a rare transition metal particle, a fluorophore, a chromophore, an enzyme, a quantum dot, and noble metal nanoparticles.

The detectable label can be a horseradish peroxidase. The detectable label can be ruthenium.

The solid support can be an electrochemiluminescence platform.

The capture reagent and/or the detection reagent can be selected from mouse anti-AbB antibodies (for example, anti-AbB-Ab1 and anti-AbB-Ab2).

The method can recover at least 50%, 55%, 60%, 65%, 75%, 80%, 85%, 90%, 95%, or 100% analyte signal in a sample comprising the interfering agent compared to a sample that does not comprise the interfering agent.

The quantity of the analyte can be determined by correlating the amount of detected detection reagent to a predetermined reference standard.

In other aspects, the inventions provide kits for detecting AbB in a sample, wherein the kits comprise the capture reagent, the detection reagent, and a dilution buffer comprising the weak acid.

In some aspects, the methods for detecting AbB in a sample comprise the steps of: (i) diluting a sample comprising AbB with a weak acid to produce an acidified sample, (ii) adding the acidified sample directly to a solid support without neutralizing the acidified sample, wherein the solid support is coated with a capture reagent, wherein the capture reagent is a first AbB-specific antibody, (iii) removing the acidified sample from the solid support after a first period of incubation, (iv) adding a detection reagent directly to the solid support, wherein the detection reagent is a second AbB-specific antibody (v) removing the detection reagent from the solid support after a second period of incubation, and (vi) detecting the detection reagent bound to AbB captured by the capture reagent, wherein the quantity of the detection reagent detected correlates to the quantity of AbB in the sample.

The capture reagent and/or the detection reagent can be selected from mouse anti-AbB antibodies (anti-AbB-Ab1 and anti-AbB-Ab2).

The assay can have an analyte recovery rate of about at least 75%, 80%, 85%, 90% or 95% in the presence of at least 0-2000 μg/mL interfering agent (for example, AbA).

The assay can detect AbB at a concentration of as low as 1.56 ng/mL in 2% human serum or 78 ng/mL in neat human serum.

In certain aspects, the inventions provide methods for detecting factor XI in a sample comprising the steps of: (i) diluting a sample comprising factor XI with a weak acid to produce an acidified sample, (ii) adding the acidified sample directly to a solid support without neutralizing the acidified sample, wherein the solid support is coated with a capture reagent that can bind to factor XI, (iii) removing the acidified sample from the solid support after a first period of incubation, (iv) adding a detection reagent directly to the solid support, (v) removing the detection reagent from the solid support after a second period of incubation, and (vi) detecting the detection reagent bound to factor XI captured by the capture reagent, wherein the quantity of the detection reagent detected correlates to the quantity of factor XI in the sample.

The methods can further comprise washing the solid support after the acidified sample is removed from the solid support. The methods can comprise washing the solid support after the detection reagent is removed from the solid support.

The sample can be a biological fluid, such as blood, serum, plasma, CSF, urine, or saliva.

The sample can be from a subject that was administered a factor XI drug product.

The factor XI drug product can comprise an antibody that binds to factor XI.

The sample can be from a subject diagnosed or suspected of having a factor XI-associated disease or disorder.

Factor XI can bind non-covalently to an interfering agent in the sample.

The interfering agent can be an antibody.

The interfering antibody can be a human anti-factor XI antibody (for example, Int-FXI-Ab1) or an factor XI-binding fragment thereof.

The acidified sample can have a pH of about 3.0-5.0, for example, about 4.1.

The weak acid can be an acid that does not completely dissociate into its ions in an aqueous solution including, but not limited to, acetic acid, citric acid, formic acid, lactic acid, phosphoric acid, and PIPES.

The sample can be acidified for 5 to 120 minutes.

The capture reagent can be an factor XI-specific antibody.

The capture reagent antibody can be a polyclonal antibody, monoclonal antibody, a bispecific antibody, an Fab fragment, an F(ab′)2 fragment, a monospecific F(ab′)2 fragment, a bispecific F(ab′)2, a trispecific F(ab′)2, a monovalent antibody, an scFv fragment, a diabody, a bispecific diabody, a trispecific diabody, an scFv-Fc, a minibody, an IgNAR, a v-NAR, an hcIgG, or a vhH.

The capture reagent can be biotinylated and the solid support is coated with streptavidin.

The capture reagent can bind directly to the solid support.

The detection reagent can be an antibody.

The detection reagent antibody can be a polyclonal antibody, monoclonal antibody, a bispecific antibody, an Fab fragment, an F(ab′)2 fragment, a monospecific F(ab′)2 fragment, a bispecific F(ab′)2, a trispecific F(ab′)2, a monovalent antibody, an scFv fragment, a diabody, a bispecific diabody, a trispecific diabody, an scFv-Fc, a minibody, an IgNAR, a v-NAR, an hcIgG, or a vhH.

The detection reagent can be conjugated to a detectable label.

The detectable label can be selected from the group consisting of a rare transition metal particle, a fluorophore, a chromophore, an enzyme, a quantum dot, and noble metal nanoparticles.

The detectable label can be a horseradish peroxidase. The detectable label can be ruthenium.

The solid support can be an electrochemiluminescence platform.

The capture reagent and/or the detection reagent can be polyclonal antibodies.

The method can recover at least 70%, 75%, 80%, 85%, 90%, 95% or 100% analyte signal in a sample comprising the interfering agent compared to a sample that does not comprise the interfering agent.

The quantity of the analyte can be determined by correlating the amount of detected detection reagent to a predetermined reference standard.

In other aspects, the inventions provide kits for detecting Factor XI in a sample, wherein the kits comprise the capture reagent, the detection reagent, and a dilution buffer comprising the weak acid.

In some aspects, the methods for detecting factor XI in a sample comprise the steps of: (i) diluting a sample comprising factor XI with a weak acid to produce an acidified sample, (ii) adding the acidified sample directly to a solid support without neutralizing the acidified sample, wherein the solid support is coated with a capture reagent, wherein the capture reagent is a first factor XI-specific antibody, (iii) removing the acidified sample from the solid support after a first period of incubation, (iv) adding a detection reagent directly to the solid support, wherein the detection reagent is a second factor XI-specific antibody (v) removing the detection reagent from the solid support after a second period of incubation, and (vi) detecting the detection reagent bound to factor XI captured by the capture reagent, wherein the quantity of the detection reagent detected correlates to the quantity of factor XI in the sample.

The assay can have an analyte recovery rate of at least 70%, 75%, 80%, 85%, 90% or 95% in the presence of 0-1500 μg/mL interfering agent (for example, a human anti-factor XI antibody, for example, Int-FXI-Ab1).

The assay can detect factor XI at a concentration of as low as 1.56 ng/mL in the presence of 2% monkey plasma, or 0.078 μg/mL in undiluted monkey plasma.

In summary, the inventions disclosed herein are advantageous over methods currently in use in the field of analyte detection because the step of neutralizing the acidified sample is not required for binding of the analyte to the capture reagent. A further advantage is that the methods of the inventions allow for sufficient direct detection and quantification of the analyte of interest in a sample subject to interference.

### V. BRIEF DESCRIPTION OF THE FIGURES

Non-limiting aspects and examples of the inventions are described with reference to figures attached hereto that are listed following this paragraph. Identical features that appear in more than one figure are generally labeled with the same label in all the figures in which they appear.

FIG. 1A is a schematic of an exemplary assay for determining the concentration of IL2Rγ in a sample. A human biotinylated anti-IL2Rγ antibody (Bio-anti-IL2Rγ-Ab1) is bound to a solid support coated with streptavidin to capture IL2Rγ. A ruthenium-labeled human anti-IL2Rγ antibody (Ru-anti-IL2Rγ-Ab2) is used to detect captured IL2Rγ. An interfering agent might be present in the assay (for example, an anti-IL2Rγ antibody, Int-IL2Rγ-Ab1).

FIG. 1B is a line graph depicting standard curve signal (counts) from IL2Rγ samples treated with acetic acid. ADB: assay dilution buffer.

FIG. 1C is a line graph depicting the percentage of recovered signal for 1 ng/mL of IL2Rγ in the presence of varying amounts of interfering agent Int-IL2Rγ-Ab1 (Int-IL2Rγ-Ab1), another human monoclonal antibody against IL2Rγ, after sample dilution in ADB containing 0 mM, 80 mM, 100 mM, 120 mM, and 150 mM acetic acid solutions.

FIG. 2A is a schematic of an exemplary assay for determining the concentration of human EGFR in a sample. An anti-EGFR antibody (R&D Systems, Cat/Part #893730) is coated directly onto a solid support. A horseradish peroxidase (HRP)-conjugated anti-EGFR antibody (R&D Systems, Cat/Part #893731) is used to detect the captured EGFR.

FIG. 2B is a line graph depicting the percentage of recovered EGFR signal in two samples (Pool 1 and Pool 2) in the presences of varying amounts of interference agent Int-EGFR-Ab1 after treatment with neutral assay dilution buffer (ADB) or ADB containing 30 mM acetic acid solution as depicted in FIG. 2A.

FIG. 3A is a schematic of an exemplary assay for determining concentration of EGFR in a sample. A biotinylated goat polyclonal anti-EGFR antibody is bound to a solid support spotted with streptavidin to capture EGFR, and ruthenium-labeled goat polyclonal anti-EGFR antibody is used to detect the captured EGFR (MSD R-PLEX Human EGFR antibody Set, Cat #F21 N5).

FIG. 3B is a line graph depicting percentage of recovered signal of EGFR in the presences of varying concentrations of interfering agent Int-EGFR-Ab1, another anti-EGFR antibody, after treatment with neutral ADB or ADB containing 30 mM acetic acid solution using the assay as depicted in FIG. 3A.

FIG. 3C is a line graph depicting signal (counts) from human EGFR samples diluted in assay dilution buffer (ADB) or ADB containing 30 mM acetic acid solution using the assay as depicted in FIG. 3A.

FIG. 3D is a bar graph showing EGFR recovery rate in the presence of 0, 31.3, 250 or 2000 μg/ml interfering agent Int-EGFR-Ab1 after treatment with ADB containing 0 mM, 5 mM, 10 mM, 15 mM, 20 mM, 25 mM, and 30 mM acetic acid using the R-PLEX acid titer based on the assay descripted in FIG. 3A.

FIG. 4A is a schematic of an exemplary assay for determining concentration of a human monoclonal antibody (IgG4 subclass) specific for natriuretic peptide receptor 1 (NPR1) (AbA) in a sample based on one example of the invention. A biotinylated mouse monoclonal antibody (Bio-anti-AbA-Ab1), which binds to the variable CDR region of AbA, is bound to a solid support spotted with streptavidin to capture AbA. Ruthenium-labeled anti-hIgG4Fc antibody (Ru-anti-hIgG4Fc), which is an anti-human IgG4 Fc-specific antibody, is used to detect captured AbA.

FIG. 4B is a line graph depicting percentage of recovered signal of AbA in the presence of an interfering agent (human monoclonal antibody against AbA: Int-AbA-Ab1, Int-AbA-Ab2; human monovalent monoclonal antibody against AbA: AbB and Int-AbA-Ab3) at a blocker:AbA ratio up to about 3,300:1 after dilution in a neutral ADB using the assay depicted in FIG. 4A.

FIG. 4C is a line graph depicting percentage of recovered signal of AbA in the presence of an interfering agent (Int-AbA-Ab1, Int-AbA-Ab2, AbB and Int-AbA-Ab3) at a blocker:AbA ratio up to about 3300:1 after dilution in ADB containing 30 mM acetic acid using the assay depicted in FIG. 4A.

FIG. 5A is a schematic of an exemplary assay for determining concentration of AbA in a sample based on another example of the invention. A biotinylated antibody (biotin-anti-AbA-Ab1) is bound to a solid support spotted with streptavidin to capture AbA. A ruthenium-labeled mouse anti-AbA monoclonal antibody (anti-AbA-Ab2) is used to detect the captured AbA. Both anti-AbA-Ab1 and anti-AbA-Ab2 bind to the variable CDR region of AbA,

FIG. 5B is a line graph depicting the percentage of recovered signal of AbA tested at both lower limit of quantitation (LLOQ) and high quality control (HQC) levels in the presence of 15.6 μg/ml to 1 mg/mL interfering agent AbB at pH 4.5 or pH 7.4 using the assay depicted in FIG. 5A.

FIG. 6A is a schematic of an exemplary assay for determining concentration of AbB, a human monovalent monoclonal antibody against the human anti-NPR1 IgG4 antibody, AbA, in a sample. An anti-AbB antibody is bound to a solid support to capture AbB. A biotinylated mouse anti-AbB antibody (Biotin-anti-AbB-Ab1) is used to detect the captured AbB.

FIG. 6B is a line graph depicting percentage of recovered signal of AbB tested at lower limit of quantitation (LLOQ) level in the presence of 31.25 μg/ml to 1 mg/mL of AbA as interfering agent at pH 4.5 or pH 7.4 using the assay depicted in FIG. 6A.

FIG. 7 is a schematic of an exemplary assay approach for determining concentration of factor XI (FXI) in a sample. An anti-FXI antibody is bound to a solid plate to capture FXI. An HRP-conjugated anti-factor XI antibody is used to detect the captured FXI.

### VI. DETAILED DESCRIPTION OF THE INVENTIONS

### A. Definitions

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are merely intended to serve as a way of referring individually to each separate value falling within the range, unless otherwise indicated herein, and each separate value is incorporated into the specification as if it were individually recited herein.

The term “about” in the context of numerical values and ranges refers to values or ranges that approximate or are close to the recited values or ranges such that the invention can perform as intended, such as having a desired rate, amount, density, degree, increase, decrease, percentage, value, purity, pH, concentration, presence of a form or variant, temperature or amount of time, as is apparent from the teachings contained herein. For example, “about” can signify values either above or below the stated value in a range of approx. +/−10% or more or less depending on the ability to perform. Thus, this term encompasses values beyond those simply resulting from systematic error.

The term “antibody,” as used herein, is an example of a binding molecule and refers to an immunoglobulin that typically comprises four polypeptide chains, two heavy (H) chains and two light (L) chains inter-connected by disulfide bonds. Each heavy chain comprises a heavy chain variable region (HCVR or VH) and a heavy chain constant region. The heavy chain constant region comprises three domains, CH1, CH2 and CH3. Each light chain contains a light chain variable region (LCVR or VL) and a light chain constant region. The light chain constant region comprises one domain (CL1). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding portion” or “antigen-binding fragment” of an antibody (or simply “antibody portion” or “antibody fragment”), as used herein, refer to one or more fragments of an antibody that retain the ability to specifically bind to an antigen (for example, IL2Rγ, EGFR, FXI). It has been shown that the antigen-binding function of an antibody can be performed by fragments of a full-length antibody. Examples of binding fragments encompassed within the term “antigen-binding portion” of an antibody include (i) an Fab fragment, a monovalent fragment comprising the VL, VH, CTI and Cn 1 domains; (ii) an F(ab′)2 fragment, a bivalent fragment comprising two F(ab)′ fragments linked by a disulfide bridge at the hinge region; (iii) a Fc fragment comprising the VH and C 111 domains; (iv) an Fv fragment comprising the VL and VH domains of a single arm of an antibody, (v) a dAb fragment (Ward, E. S., et al., Nature 241:544-546 (1989)), which comprises a VH domain; and (vi) a CDR. Furthermore, although the two domains of the Fv fragment, VL and VH, are coded for by separate genes, they can be joined, using recombinant methods, by a synthetic linker that enables them to be made as a single contiguous chain in which the VL and VH regions pair to form monovalent molecules (known as single chain Fv (scFv); see for example, Bird, R. E., et al, Science 242:423-426 (1988); and Huston, J. S., et al, Proc. Natl. Acad. Sci. USA 85:5879-5883 (1988)). Such single chain antibodies are also intended to be encompassed within the term “antigen-binding portion” of an antibody. Other forms of single chain antibodies, such as diabodies, are also encompassed (see for example, Holliger, P., et al, Proc. Natl. Acad Sci. USA 90:6444-6448 (1993)).

The term “binding molecule,” as used herein, is intended to refer to molecules that specifically interact with and bind to a particular target. The target can comprise a biologic or small (chemical) molecule. The target molecule may define an antigen or antigenic moiety. Examples of a binding molecule include, but are not limited to, antibodies (including polyclonal antibodies, monoclonal antibodies, bispecific antibodies, as well as antibody fragments), fusion proteins, and other antigen-binding molecule known to those skilled in the art. A binding molecule can be used as a capture reagent, detection reagent, or both in the assay of the invention.

A “CDR” or complementarity determining region is a region of hypervariability interspersed within regions that are more conserved, termed “framework regions” (FR). The FRs may be identical to the human germline sequences, or may be naturally or artificially modified.

The term “epitope” is an antigenic determinant that interacts with a specific antigen binding site in the variable region of an antibody molecule known as a paratope. A single antigen may have more than one epitope. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sufonyl groups on the antigen.

The terms “individual,” “subject,” and “patient” are used interchangeably herein, and refer to a mammal, including, but not limited to, humans, rodents, such as mice and rats, and other laboratory animals.

The terms “interfering agent” and “assay interference” refer to endogenous and/or exogenous components in the assay that block or inhibit detection of the analyte, such as IL2Rγ, EGFR, a human monoclonal antibody against NPR1 (for example, AbA), a human monovalent monoclonal antibody against AbA (AbB), and FXI, or an anti-IL2Rγ, EGFR, AbA, AbB, and FXI antibody. Substances that alter the measurable concentration of the analyte or alter antibody binding can potentially result in immunoassay interference. An interfering agent may bind directly to the analyte, or may be part of a complex that comprise the analyte. A complex that comprises the analyte and an interfering agent is referred to herein as “interfering agent:analyte complex” or “analyte:interfering agent complex”. Assay interference may be analyte-dependent or -independent. Analyte-independent interferences refer to the common interferences of hemolysis, lipemia and effects of anticoagulant and sample storage, which are independent of the analyte concentration. Analyte-dependent interference in the immunoassays refers to interactions between an interfering agent with the analyte, directly or indirectly, or between an interfering agent with the analyte-specific antibody that block or inhibit detection of the analyte. Interfering agent may be compounds with chemical differences but structural similarities that cross-react with the antibody. Interfering and endogenous substances that are natural, polyreactive antibodies or autoantibodies (heterophiles), human anti-animal antibodies, or anti-drug antibodies (ADAs) together with other unsuspected binding proteins that are unique to the individual, can interfere with the reaction between analyte and reagent antibodies in an immunoassay.

Interference can be caused by soluble binding targets of the analyte, endogenous ligands of the analyte including but not limited to soluble receptors of the analyte, soluble ligands of the analyte, shed receptors of the analyte, or serum factors such as rheumatoid factor and biotin. Interference can be caused by antibodies to the analyte.

The term “weak acid” refers to an acid that does not completely dissociate into its ions in an aqueous solution. Most organic acids are weak acids. The strength of an acid can be quantified by its acid dissociation constant, Kₐ value. Some exemplary weak acids are acetic acid, ascorbic acid, benzoic acid, boric acid, citric acid, formic acid, hydrazidic acid, hydrocyanic acid, hydrofluoric acid, hypochlorous acid, lactic acid, nitrous acid, oxalic acid, phenolic acid, propanic acid, sulfurous acid, uric acid, phosphoric acid, and PIPES (piperazine-N,N-bis(2-ethanesulfonic acid).

The term “analyte” or “analytes” refers to the substance whose presence is intended to be quantitatively analyzed. The analytes could be a ligand that specifically bind or ligate to both an immobilized capture agent and a detection agent. The analytes could a substance such as peptides, proteins, antibodies, and hormones presented in a biological fluid that plays an important role on biological functions.

The term “analyte recovery (% AR)” or “recovery rate” refers to the fraction of the analyte detected in a sample (for example, a sample that contains an interfering agent) compared to the actual amount or concentration of the analyte in the sample. In other words, analyte recovery is the percentage of the measured analyte concentration (concentration or mean concentration) as a fraction of the nominal (spiked) concentration.

### B. Detection and Quantification Assays, Kits and Methods of Use Thereof

a. Methods

The present inventions relate to assays and methods for the detection and quantification of an analyte. The disclosed assays and methods can be used to detect and quantify a protein analyte (for example, IL2Rγ, EGFR, a human monoclonal antibody against NPR1 (AbA), a human monovalent monoclonal antibody against AbA (AbB), FXI) in a sample. The sample can be obtained from an individual treated with a drug product or is subject to a medical treatment.

The disclosed assays have reduced assay interference relative to the commercially available assays and/or a control assay. The interference can be analyte-dependent, analyte-independent, or both. The methods can reduce or inhibit assay interference caused by endogenous soluble analyte binding molecules present in the sample. The endogenous soluble analytes binding molecules include, but are not limited to serum components, anti-target analyte-antibodies, and target analyte receptors.

The methods and assays of the inventions are for detecting an analyte in a sample by acidifying the sample to a pH sufficient to dissociate analyte from its complexes in the sample. Capture and detection reagents that bind specifically to the analyte are used to detect and quantify the analyte in the sample, for example, determine the amount or concentration of the analyte in the sample.

The methods and assays of the inventions are also useful for decreasing assay interference by acidifying samples to a pH sufficient to dissociate an analyte from its complexes in the sample. The methods comprise acidifying a capture reagent and sample mixture to a pH sufficient to dissociate interfering agent from the analyte and enable binding of the analyte and capture reagent, immobilizing the capture reagent to a solid support, adding a detection reagent, optionally wherein the detection reagent comprises a detectable label, and allowing the detection reagent to bind to the analyte captured by the immobilized capture reagent. The quantity of detection reagent correlates to the quantity of captured analyte in the sample.

In certain aspects, the methods and assays of the inventions are provided to decrease analyte interference in a sample by acidifying the sample to a pH sufficient to dissociate the analyte from its complexes in the sample. The methods include adding the acidified sample to a capture reagent immobilized to a solid support, adding a detection reagent, optionally wherein the detection reagent comprises a detectable label, and allowing the detection reagent to bind to the analyte captured by the capture reagent, wherein the quantity of detectable label detected correlates to the quantity of the analyte in the sample.

In certain aspects, the methods and assays of the inventions are provided for quantifying an analyte in a sample by acidifying the sample to a pH sufficient to dissociate the analyte from its complexes in the sample. The methods include adding the acidified sample to a capture reagent immobilized to a solid support, adding a detection reagent, optionally wherein the detection reagent comprises a detectable label, and allowing the detection reagent to bind to the analyte captured by the capture reagent, wherein the quantity of detectable label detected correlates to the quantity of the analyte in the sample.

The methods and assays can be used to determine the concentration of an analyte in human serum samples using an electrochemiluminescence immunoassay. The methods include acid pre-treatment of serum samples to dissociate soluble analyte: interfering agent complexes present in the samples and improve detection of the analyte in the presence of the interfering agent, thus providing a quantitative measurement of the levels of the analyte.

The amount of label detected in the sample can be compared to a reference standard calibrated with known concentrations of the analyte and corresponding amounts of detected label for the concentrations. The amount of analyte in the sample can be determined by comparing the amount of detected label in the sample to the reference standard and matching the amount of detected label with the concentration shown on the reference standard for that amount of detected label.

The methods and assays can employ a streptavidin-coated plate, with a biotinylated antibody as the capture reagent, and utilizes recombinant analyte as a standard. The standards, controls, and samples are diluted in dilution buffer containing acetic acid. The detection reagent is a ruthenium-labeled antibody. The analyte captured on the plate is measured by a chemiluminescent signal generated by the ruthenium label when voltage is applied to the plate by the plate reader. The resulting electrochemiluminescent signal (for example, counts) is proportional to the amount of analyte present in the samples.

The capture reagent can be pre-coated to a solid support, for example, a solid support spotted with streptavidin, to capture the analyte-of-interest in an acidified sample. The capture antibody can be mixed with sample containing analyte-of-interest first and acidified for a period of time, for example, about 1 hour, before being added to a blocked assay plate for further detection.

The analytes can be treated with assay dilution buffer (ADB) containing different concentrations of mild acid, for example, 20 mM, 30 mM, 60 mM, 80 mM, 100 mM, 120 mM, or 150 mM acetic acid, to bring the pH level to 6.5-3.0, in order to efficiently dissociate the interfering agent:analyte complex.

The interfering agent can be completely dissociated from the analyte after sample acidification. The interfering agent can be partially dissociated from the analyte after sample acidification.

About 50%-100% of the analyte in the sample can be dissociated from the interfering agent:analyte complex. About at least 50%, 55%, 60%, 65%, 70%, 75%, 80%, 85%, 90%, 95% or 100% of the analyte in the analyte can be dissociated from the interfering agent:analyte complex.

The methods can further comprise the step of determining if the interfering agent dissociates from the analyte after sample acidification. The methods can comprise testing different pH levels to determine at which mildly acidic pH the interfering agent dissociates from the analyte. The methods can comprise testing different pH levels to determine at which mildly acidic pH the interfering agent dissociates from the analyte. The methods can comprise testing different pH levels to determine at which mildly acidic pH the interfering agent does not bind to the analyte, or has reduced binding to the analyte compared to the binding at the original pH of the sample. Methods to measure interactions between two molecules are well known in the art (for example, surface plasmon resonance) and can be used to determine binding and dissociation of the interfering agent and the analyte at different pH conditions.

A working pH useful for the methods and assays of the invention is a pH level where the interfering agent dissociates partially or completely from the analyte. At least 50%, 55%, 60%, 65%, 70%, 75%, 80%, 85%, 90%, 95%, or 100% of the interfering agent bound to the analyte in the unacidified sample can dissociate from the analyte after sample acidification.

The methods can further comprise the step of identifying a capture agent that can bind to the analyte at the pH of the sample after acidification. The methods can comprise screening for a capture agent that can bind to the analyte at the pH where the interfering agent dissociate or has reduced binding to the analyte.

Methods to measure interactions between two molecules are well known in the art (for example, surface plasmon resonance) and can be used to determine binding of a candidate capture reagent and the analyte at different pH conditions.

The sensitivity of the assay, which is determined by the lower limit of quantitation (i.e., lower detection limit) of the analyte, can be determined at different sample acidification conditions. The tolerance of an analyte to one or more interfering agents can be also determined under different sample acidification conditions.

The methods can have a lower limit of quantitation (LLOQ) (that is, the minimal amount of analyte required to be in the sample to be detectable by the method) of 0.5-20 pg/mL, 1-50 pg/mL, 5-200 pg/mL, 20-400 pg/mL, 0.1-0.5 ng/ml, 0.2-0.8 ng/mL, 0.1-1 ng/mL, 0.5-2 ng/mL, 1-10 ng/mL, 5-50 ng/mL, 20-60 ng/mL, 40-100 ng/mL, 50-150 ng/mL, or 100-200 ng/mL. The methods can have a lower limit of quantitation (LLOQ) of less than 0.5 pg/mL, 1 pg/mL, 5 pg/mL, 10 pg/mL, 15 pg/mL, 20 pg/mL, 40 pg/mL, 60 pg/mL, 80 pg/mL, 0.1 ng/mL, 0.5 ng/ml, 0.8 ng/mL, 1 ng/mL, 1.5 ng/mL, 2 ng/mL, 5 ng/mL, 10 ng/mL 15 ng/mL, 20 ng/mL, 30 ng/mL, 40 ng/mL, 50 ng/mL, 60 ng/mL, 70 ng/mL, 80 ng/mL, 90 ng/mL, 100 ng/mL, 150 ng/mL, 200 ng/mL, 300 ng/mL, 500 ng/mL, 750 ng/mL, 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 15 pg/mL, 20 pg/mL, 25 pg/mL, 30 pg/mL, 35 pg/mL, 40 pg/mL, 45 pg/mL, or 50 pg/mL, 60 pg/mL, 70 pg/mL.

The assay can be carried out in serum free condition. The assay can be carried in the presence of serum (for example, human serum, bovine serum, monkey serum or horse serum). The assay can be carried out in undiluted (for example, neat) serum.

The methods can recover at least 50%, 55%, 60%, 65%, 70%, 75%, 80%, 85%, 90%, 95% or 100% analyte signal in a sample comprising the interfering agent as compared to a sample that does not comprise the interfering agent.

The methods can be carried out at about 4-37° C., for example, at about 4° C. The methods can be carried out at about 20-37° C., for example, 20-25° C., 25-30° C., or 30-37° C. For example, the methods can be carried out at about 20° C., 25° C., 30° C., or 37° C.

The acidified sample can be added directly to the capture reagent without being neutralized with a buffered basic solution.

A blocking agent can be further added to the sample to reduce or prevent the reformation of the interfering agent:analytes complex after sample acidification.

b. Samples

The sample used in the disclosed methods is typically a biological sample such as a biological fluid containing the analyte to be detected. Biological fluids include, but are not limited to blood, plasma, serum, saliva, cerebrospinal fluid (CSF), and urine.

The sample can be obtained from a subject having or suspected of having a disorder or disease.

The sample can be from a subject that was administered a drug product.

The sample can be from a subject that has substance abuse or is addicted to alcohol and tobacco products.

The sample can be from a subject that has having or suspected of having been exposed to a toxin, an allergen, or an irritant.

The analyte to be detected is typically a component of serum taken from a subject, for example, a human subject. Representative analytes to be detected and/or quantified in the sample include, but are not limited to, cytokines, protein drug products, and metabolites or fragments thereof.

Representative protein drug products include, but are not limited to recombinant proteins, antibodies, and fusion proteins. The antibodies can be polyreactive antibodies or autoantibodies (heterophiles), human anti-animal antibodies, or anti-drug antibodies.

c. Acidification of the Samples

The sample in the disclosed methods can be acidified for 1 to 120 minutes. The sample can be acidified for about 1, 2, 3, 4, 5, 6, 7, 8, 9, 10, 11, 12, 13,14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 minutes. The sample can be acidified for about 65, 70, 75, 80, 85, 90, 95, 100, 105, 110, 115 or 120 minutes.

Acids that can be used in the acidification step include, but are not limited to, acetic acid, citric acid, formic acid, lactic acid, phosphoric acid and PIPES.

The sample can be acidified to a pH to dissociate an analyte from its complexes with one or more interfering agents, for example, pH of 3.0 to 6.5. The pH can be about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, or 6.5. The analyte can be completely dissociated from the complexes with the interfering agents at the pH of the sample after acidification. The analyte can be partially dissociated from the complexes with the interfering agents at the pH after sample acidification.

d. Capture Reagents

The capture reagents or capture agents described herein include molecules that bind to the analytes, for example, analyte antagonists or inhibitors, such as antibodies or antigen-binding fragments of antibodies that specifically bind to the analytes. The antibodies can be monoclonal, polyclonal, or humanized. The term “specifically bind,” or the like, means that the antibodies or antigen-binding fragments thereof forms a relatively stable complex with the antigens. The capture reagents of the inventions bind to their corresponding analytes at the pH of the acidified sample, i.e., the pH of the acidified sample does not significantly reduce binding affinity of the capture reagents for the analytes. These capture reagents could be either monoclonal or polyclonal antibodies raised in different animal species.

The capture reagent can be biotinylated. The capture reagent can be fixed to or linked to a solid support. The solid support can be a microplate, for example, a streptavidin-coated microplate.

The capture reagent can bind the analyte with a dissociation constant (K_{D}) value of ≤1 μM, ≤100 nM, ≤50 nM, ≤25 nM, ≤20 nM, ≤15 nM, ≤10 nM, ≤5 nM, ≤2 nM, ≤1 nM, ≤0.1 nM, ≤0.01 nM, or ≤0.001 nM (for example, 10⁻⁸ M or less, for example, from 10⁻⁸ M to 10⁻¹³ M, for example, from 10⁻⁹ M to 10⁻¹³ M).

The capture reagent can bind to IL2Rγ, EGFR, human monoclonal antibody against NPR1 (AbA), human monovalent monoclonal antibody against AbA (AbB), or FXI for use in methods or assays for measuring IL2Rγ, EGFR, AbA, AbB, FXI in a sample, respectively.

The capture reagent in the disclosed methods and assays can be biotinylated anti-AbA-Ab1, a mouse anti-AbA monoclonal antibody. The capture reagent can be an EGFR antibody or biotinylated anti-EGFR antibody, a mouse anti-AbB antibody (anti-AbB-Ab2), or a goat anti-human FXI polyclonal antibody.

e. Detection Reagents

The detection reagents and detection agents described herein includes molecules that bind to the analyte, for example, an analyte antagonist or inhibitor, for example, an analyte antibody or antigen-binding fragments of antibodies that specifically bind to the analytes. The antibodies can be monoclonal, polyclonal, or humanized.

The detection reagent can be labeled with a detectable label. Detectable labels are known in the art and include, but are not limited to a rare transition metal particle, a fluorophore, a chromophore, a quantum dot, noble metal nanoparticles, a radioactive moiety, an enzyme, a bioin/avidin label, and chemiluminescent label. The detection reagent can be ruthenylated anti-AbA-Ab2, a mouse anti-AbA monoclonal antibody. The detection reagent can be a biotinylated-mouse anti-AbB antibody (anti-AbB-Ab1), a goat anti-human FXI polyclonal antibody conjugated to peroxidase (HRP), a HRP-conjugated anti-EGFR antibody, or a ruthenium-labeled anti-EGFR antibody.

The detection reagent can bind the analyte with a dissociation constant (K_{D}) value of 1 μM, ≤100 nM, ≤50 nM, ≤25 nM, ≤20 nM, ≤15 nM, ≤10 nM, ≤5 nM, ≤2 nM, ≤1 nM, ≤0.1 nM, ≤0.01 nM, or ≤0.001 nM (for example, 10-8 M or less, for example, from 10⁻⁸ M to 10⁻¹³ M, for example, from 10-9 M to 10⁻¹³ M).

The detection reagent can bind to IL2Rγ, EGFR, AbA, AbB, or FXI for use in methods or assays for measuring IL2Rγ, EGFR, AbA, AbB, or FXI in a sample, respectively.

f. Kits

Kits for assaying a test sample for the presence, amount or concentration of an analyte (or a fragment thereof) in a sample are also provided. The kits include at least one component for assaying the sample for the analyte (or a fragment thereof) and instructions for assaying the sample for the analyte (or a fragment thereof).

The kits of the invention include a detection reagent as described above, a capture reagent as described above.

The detection reagent and the capture reagent can be the same or different analyte binding molecules as described above, such as a monoclonal antibody (or a fragment, a variant, or a fragment of a variant thereof), a fusion protein, or an apatamer optionally immobilized on a solid phase.

After the capture reagent binds to the analyte, the binding site and the epitope of the detection reagent can still be available for the detection reagent to bind to the analyte.

The capture reagent and the detection reagent can bind to different epitopes of the analyte.

Binding of the capture reagent to the analyte may not hinder binding of the detection reagent to the analyte. For example, the epitopes of the capture reagent and detection reagent are distant and therefore binding of one reagent does not sterically hinder binding of the other reagent.

The detection reagent is typically labeled with a detectable label such as a chemiluminescent label. The detection reagent can incorporate a detectable label as described herein, such as a fluorophore, a radioactive moiety, an enzyme, a biotin/avidin label, a chromophore, a chemiluminescent label, or the like, or the kit can include reagents for carrying out detectable labeling. The antibodies, calibrators and/or controls can be provided in separate containers or pre-dispensed into an appropriate assay format, for example, into microtiter plates. The analyte detection reagent can be labeled with ruthenium. The detection reagent can be conjugated with HRP.

The kits can contain a biotin labeled capture reagent and a streptavidin coated solid support, for example, a microtiter plate or electrochemiluminescence platform. The capture reagent and the microtiter plate or electrochemiluminescence platform can be provided in separate containers. The kits can contain a microtiter or electrochemiluminescence platform plate coated with the capture reagent.

The kits can also contain acid solutions and buffers as described above for treating the samples.

The kits can include a calibrator or control, for example, isolated, natural and/or recombinant analyte. The kits can include at least one container (for example, tube, microtiter plates or strips or electrochemiluminescence platform) for conducting the assay, and/or a buffer, such as an assay buffer or a wash buffer, either one of which can be provided as a concentrated solution, a substrate solution for the detectable label (for example, an enzymatic label), or a stop solution. Preferably, the kits include all components, i.e., reagents, standards, buffers, diluents, etc., which are necessary to perform the assay. The instructions can be in paper form or computer-readable form.

Optionally, the kits include quality control components (for example, sensitivity panels, calibrators, and positive controls). Preparation of quality control reagents is well known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kits can also optionally include other reagents required to conduct a diagnostic assay or facilitate quality control evaluations, such as buffers, salts, enzymes, enzyme co factors, enzyme substrates, detection reagents, and the like. Other components, such as buffers and solutions for the isolation and/or treatment of a test sample (for example, pretreatment reagents), also can be included in the kits. The kits can additionally include one or more other controls. One or more of the components of the kits can be lyophilized, in which case the kits can further comprise reagents suitable for the reconstitution of the lyophilized components.

The various components of the kits are optionally provided in suitable containers as necessary, for example, a microtiter plate. The kits can further include containers for holding or storing a sample (for example, a container or cartridge for a urine sample). Where appropriate, the kits optionally can also contain reaction vessels, mixing vessels, and other components that facilitate the preparation of reagents or the test sample. The kits can also include one or more instruments for assisting with obtaining a test sample, such as a syringe, pipette, forceps, measured spoon, or the like.

### C. Methods and Assays for Detection and Quantification of IL2Rγ

a. Interleukin-2 Receptor γ-Chain

IL2Rγ or “Interleukin-2 receptor γ-chain” refers to the 64 kDa IL2Rγ transmembrane protein with 347 amino acids, 84 of which are cytoplasmic. IL2Rγ plays a pivotal role in formation of the full-fledged IL-2 receptor that are important for T cell differentiation, activation, expansion, and survival. Together with the beta chain, IL2Rγ participates in increasing the IL-2 binding affinity and intracellular signal transduction. IL2Rγ is also a shared component of the receptor complexes for cytokines IL-2, IL-4, IL-7, IL-9, and IL-15. Multiple pathways downstream of these receptors are essential for differentiation and growth of T cells and NK cells, which collectively have actions related to the control of cancer, autoimmune diseases, and immunodeficiency, and as such are of substantial clinical interest. IL2Rγ mutations is associated with λ-linked severe combined immunodeficiency in humans, a disease characterized by the presence of few or no T cells.

b. Methods and Kits

The methods and kits described herein are intended for the quantitative determination of IL2Rγ or a fragment thereof in a sample, for example, natural and/or recombinant IL2Rγ, by acidifying the sample to a pH sufficient to dissociate IL2Rγ from its complexes in the sample. Capture and detection reagents specific for IL2Rγ are used in the methods and kits to detect and determine the amount or concentration of IL2Rγ in the sample.

The methods and kits described herein also provide capture reagent and detection reagent combination for IL2Rγ detection and quantification, wherein after the capture reagent binds to IL2Rγ, the binding site and the epitope of the detection reagent are available for the detection reagent to bind to IL2Rγ.

The capture reagent can be an anti-IL2Rγ antibody or antigen-binding fragment thereof. The detection reagent can be an anti-IL2Rγ antibody or antigen-binding fragment thereof.

Anti-IL2Rγ antibodies as well as methods of making anti-IL2Rγ antibodies can be known in the art. For example, descriptions of a number of anti-IL2Rγ antibodies may be found in patent publication US2020/0247894A1.

The capture reagent can be an anti-IL2Rγ antibody disclosed in US2020/0247894 A1. The capture reagent can be anti-IL2Rγ-Ab2 or anti-IL2Rγ-Ab1. The detection reagent can be an anti-IL2Rγ antibody disclosed in US2020/0247894 A1. The detection reagent can be anti-IL2Rγ-Ab2 or anti-IL2Rγ-Ab1.

The methods disclosed herein can recover at least 70-100% signal of the IL2Rγ, i.e., have a recovery rate of at least 50%, 55%, 60%, 65%, 70%, 75%, 80%, 85%, 90%, 95% or 100%.

The sample can be acidified to a pH of about 4.0-5.0, for example, a pH of about 4.0, 4.1, 4.2, 4.3, 4.4, 4.5, 4.6, 4.7, 4.8, 4.9 or 5.0.

In some exemplary and non-limiting assays, IL2Rγ is detected and quantified in a biological sample including serum, plasma, saliva, CSF, and urine as described herein. The assay employs a streptavidin microplate coated with biotinylated anti-IL2Rγ antibody (bio-anti-IL2Rγ-Ab1) as a capture agent, and utilizes IL2Rγ as standards. Standards, controls and samples are diluted in assay dilution buffer (ADB) prior to addition to the assay plate. Captured IL2Rγ is then detected using ruthenium-labeled IL2Rγ detection antibody (Ru-anti-IL2Rγ-Ab2). An electrochemiluminescent signal is then generated by the ruthenium label when voltage is applied to the plate by a reader. The measured electrochemiluminescence (i.e., counts) is proportional to the concentration of total IL2Rγ in the samples. The samples may contain an interfering reagent, for example, human anti-IL2Rγ antibody, Int-IL2Rγ-Ab1.

The inventions also provide methods for evaluating the effect of sample acidification on assay sensitivity in IL2Rγ quantification. The methods comprise incubating IL2Rγ-containing sample with neutral or acidic assay dilution buffer (ADB) to acidify the sample, for example, lower the pH of the sample to 4.4-4.0, detecting IL2Rγ using the methods and assays of the inventions as described in this disclosure, and comparing the detected value of IL2Rγ in an acidified sample to the value in a neutral sample.

The inventions also provide methods for assessing IL2Rγ signal recovery rate in the presence of an interfering agent, for example, Int-IL2Rγ-Ab1. Samples comprising only IL2Rγ or IL2Rγ and one or more interfering agents (for example, Int-IL2Rγ-Ab1) are diluted with a neutral or acidic assay dilution buffer (ADB) to acidify the sample, for example, lower the pH of the sample to 4.4-4.0. IL2Rγ signals detected from the samples comprising the interfering agents are compared to signals detected from the samples that do not comprise the interfering agents to determine the analyte recovery rate.

### D. Methods and Assays for Detection and Quantification of EGFR

a. Epidermal Growth Factor Receptor (EGFR)

EGFR or “human epidermal growth factor receptors” or ErbB1 or HER1 refers to a 170 kDa type 1 transmembrane glycoproteins encoded by the EGFR gene located on chromosome 7. EGFR plays essential role in regulating cell proliferation, survival, differentiation and migration. The human epidermal growth factor receptors of receptor tyrosine kinases (RTK) consists of four members: EGFR (HER1), HER2, HER3, HER4. EGFR is activated by binding to its cognate ligands such as EGF (Epidermal Growth Factor) and TGF alpha (Transforming Growth Factor alpha) to the extracellular domain, which leads to EGFR dimerization followed by autophosphorylation of the tyrosine residues in the cytoplasmic domain. Phosphorylation of EGFR at certain residues is mediated by Src-non-receptor kinase. EGFR activation signals multiple downstream signaling cascades including Ras/MARK, PLCY1/PCK, PI3K/AKT, and STAT pathways that help in growth and proliferation of cells. Phosphorylation of EGFR at Y1086 specifically allows binding of the adaptor protein GRB2, leading to activation of the MAPK pathway. Upon receptor activation and signaling, EGFR is endocytosed and targeted for degradation or recycling. Soluble EGFR consists of the extra-cellular domain of EGFR, which can be measured directly in biological fluids like serum or plasma. EGFR overexpression has been associated with uncontrolled tumor growth in the head and neck, brain, bladder, stomach, breast, lung, endometrium, cervix, vulva, ovary, esophagus, stomach and in squamous cell carcinoma. Therefore, the detection and measurement of EGFR in a sample, such as human serum, provide an important method for disease diagnosis and therapeutic drug monitoring.

b. Methods

The assays described herein are intended for the quantitative determination of soluble EGFR or a fragment thereof in a sample including, for example, natural and/or recombinant EGFR, by acidifying the sample to a pH sufficient to dissociate EGFR from its complexes in the sample. Capture and detection reagents specific for EGFR are used in the methods and kits to detect and determine the amount or concentration of EGFR in the sample.

The methods and kits described herein also provide capture reagent and detection reagent combinations for EGFR detection and quantification, wherein after the capture reagent binds to EGFR, the binding site and the epitope of the detection reagent are available for the detection reagent to bind to EGFR.

The capture reagent can be an anti-EGFR antibody or antigen-binding fragment thereof. The detection reagent can be an anti-EGFR antibody or antigen-binding fragment thereof.

Anti-EGFR antibodies and methods of making anti-EGFR antibodies are known in the art. For example, descriptions of a number of anti-EGFR antibodies or antigen-binding fragments thereof are disclosed in patent publications WO 2014/004427 and WO 2020/198009.

An interfering agent may be present in the sample. The interfering agent may be a molecule administered to a subject as a drug that targets EGFR, for example, a human anti-EGFR antibody disclosed in WO 2020/198009.

The methods can recover at least 50% to 100% signal of EGFR, i.e., have a recovery rate of at least 50%, 55%, 60%, 65%, 70%, 75%, 80%, 85%, 90%, 95% or 100%.

The sample can be acidified to a pH of about 4.0-5.0, for example, a pH of about 4.0, 4.1, 4.2, 4.3, 4.4, 4.5, 4.6, 4.7, 4.8, 4.9 or 5.0.

The sample acidification can be accomplished by the addition of an assay dilution buffer comprising 30 mM, 25 mM, 20 mM, 15 mM, 10 mM, or 5 mM acetic acid.

In some exemplary and non-limiting assays for determining the concentration of total soluble EGFR in a sample, an EGFR capture antibody is pre-coated to a solid support to capture the soluble EGFR in a sample. A HRP-conjugated EGFR detection antibody is used to detect the captured EGFR, A peroxidase-specific substrate is then added to generate a signal intensity that is proportional to the amount and concentration of EGFR in a sample.

In other exemplary and non-limiting assays, a streptavidin microplate is coated with a biotinylated EGFR capture antibody, and EGFR captured on the plate is detected using a ruthenium-labeled EGFR detection antibody. Recombinant EGFR is used as a standard. Standards, controls and samples are diluted in the acidic buffer prior to addition to the assay plate. An electrochemiluminescent signal is generated by the ruthenium label when voltage is applied to the plate by a reader. The measured electrochemiluminescence (i.e., counts) is proportional to the amount and concentration of EGFR in the samples.

It is understood that one or both assay formats could be used for any of the examples disclosed herein.

The recovery rate of EGFR in the presence of an interfering agent, for example, a bispecific human anti-EGFR antibody such as Int-EGFR-Ab1, can be evaluated in a neutral or an acidic assay dilution buffer using the methods described above.

The inventions also provide methods for evaluating the effect of sample acidification on assay sensitivity in EGFR quantification. The methods comprise incubating in EGFR-containing sample with neutral or acidic assay dilution buffer (ADB) to acidify the sample, for example, lower the pH of the sample to 4.4-4.0, detecting in EGFR using the methods and assays of the inventions as described in this disclosure, and comparing the detected value of in EGFR in an acidified sample to the value in a neutral sample (for example, pH of 7.4).

The inventions also provide methods for assessing EGFR signal recovery rate in the presence of an interfering agent, for example, a bispecific human anti-EGFR antibody such as Int-EGFR-Ab1. Samples comprising only EGFR or EGFR and one or more interfering agents (for example, Int-EGFR-Ab1) are diluted with a neutral or acidic assay dilution buffer (ADB) to acidify the sample, for example, to lower the pH of the sample to 4.4-4.0. EGFR signals detected from the samples comprising the interfering agent are compared to signals detected from the samples that do not comprise the interfering agent to determine the analyte recovery rate.

### E. Methods and Assays for Detection and Quantification of Antibody Targeting a Human Monoclonal Antibody (IgG4 Subclass) Specific for NPR1 (AbA)

a. Human Monoclonal Antibody (IgG4 Subclass) Specific for NPR1 (AbA)

Natriuretic peptide receptor 1 (NPR1) is the primary receptor for natrietic peptides ANP and BNP. Binding of ANP to the extracellular ligand binding domain and of ATP to the intracellular kinase homology domain activates a cytoplasmic guanylate cyclase. AbA is a human antibody (IgG4 subclass) specific for natriuretic peptide receptor 1 (NPR1) and a drug candidate.

b. Methods

The assays described herein are intended for the quantitative determination of AbA in a sample by acidifying the sample to a pH sufficient to dissociate AbA from its complexes in the sample. Capture and detection reagents specific for AbA are used in the methods and kits to detect and determine the amount or concentration of AbA in the sample.

The methods and kits described herein also provide capture reagent and detection reagent combinations for AbA detection and quantification, wherein after the capture reagent binds to AbA, the binding site and the epitope of the detection reagent are available for the detection reagent to bind to AbA.

The capture reagent can be an anti-AbA antibody or antigen-binding fragment thereof. The detection reagent can be an anti-AbA antibody or antigen-binding fragment thereof.

The capture agent can be a mouse anti-AbA monoclonal antibody (for example, anti-AbA-Ab1). The detection reagent can be selected from an anti-human IgG4 Fc monoclonal antibody (for example, anti-hIgG4Fc) or a mouse anti-AbA monoclonal antibody (for example, anti-AbA-Ab2). An example of an anti-human IgG4 Fc antibody can be found in Mol Cancer Ther (2019) 18 (11): 2051-2062.

One or more interfering agents may be present in the assay, for example, AbA reversal agents that are human anti-AbA monoclonal antibodies (Int-AbA-Ab1, Int-AbA-Ab2, AbB, and Int-AbA-Ab3).

The sample acidification can recover at least 50%, 55%, 60%, 65%, 70%-100% signal of the analyte, i.e., has a recovery rate of at least about 70%, 75%, 80%, 85%, 90%, 95% or 100%.

The sample can be acidified to a pH of about 4.0-5.0, for example, a pH of about 4.0, 4.1, 4.2, 4.3, 4.4, 4.5, 4.6, 4.7, 4.8, 4.9 or 5.0.

Some exemplary and non-limiting assay formats relate to determining the concentration of total AbA in a sample by immobilizing a biotinylated AbA capture reagent to an avidin-coated plate to capture the total AbA, and detecting the captured AbA with a ruthenium-conjugated anti-human IgG4 Fc antibody (anti-hIgG4Fc). An electrochemiluminescent signal is then generated by the ruthenium label when voltage is applied to the plate by a reader. The measured eletrochemiluminescence is proportional to the concentration of total AbA in the samples.

In other exemplary and non-limiting assay formats, a streptavidin microplate is coated with biotinylated mouse anti-AbA monoclonal antibody anti-AbA-Ab1 as a capture agent, and a ruthenylated mouse anti-AbA monoclonal antibody, REGN1049, is used as a detection agent. The concentration of AbA is determined as described above.

It is understood that one or more assay formats could be used for any of the inventions disclosed herein.

The inventions also provide methods for evaluating the effect of sample acidification on assay sensitivity in AbA quantification. The methods comprise incubating a AbA-containing sample with neutral or acidic assay dilution buffer (ADB) to acidify the sample, for example, lower the pH of the sample to 5.0-4.0, detecting AbA using the methods and assays of the inventions as described in this disclosure, and comparing the detected value of in EGFR in an acidified sample to the value in a neutral sample (for example, pH of 7.4).

The inventions also provide methods for assessing in AbA signal recovery rate in the presence of one or more interfering agents, for example, Int-AbA-Ab1, Int-AbA-Ab2, AbB, or Int-AbA-Ab3. Samples comprising only AbA or AbA and one or more interfering agents (for example, Int-AbA-Ab1, Int-AbA-Ab2, AbB, or Int-AbA-Ab3) are diluted with a neutral or acidic assay dilution buffer (ADB) to acidify the sample, for example, lower the pH of the sample to 5.0-4.0. AbA signals detected from the samples comprising the interfering agent are compared to signals detected from the samples that do not comprise the interfering agent to determine the analyte recovery rate.

In some exemplary and non-limiting examples, AbA recovery rate in the presence of one or more of the interfering agents, for example, Int-AbA-Ab1, Int-AbA-Ab2, AbB, and Int-AbA-Ab3, is measured in both neutral assay dilution buffer and acidic assay dilution buffer, for example, assay dilution buffer containing 30 mM acetic acid. AbA tolerance to the interfering agent is assessed by the addition of varying amounts of the interfering agent to reach an interfering agent:AbA ratio ranging from 0 to 3,300:1.

The recovery rate of AbA at a concentration of 13.7 ng/mL-7.5 μg/mL, for example, at 13.7 ng/mL and 7.5 μg/mL in the presence of varying amounts of AbB can be at least 55-100%, for example, at least 50%, 55%, 60%, 65%, 70%, 75%, 80%, 85%, 90%, 95% or 100%.

### F. Method and Assays for Detection and Quantification of a Reversal Agent of Human Monoclonal IgG4 Antibody Against NPR1

a. Reversal Agent of Human Monoclonal IgG4 Antibody Against NPR1 (AbB)

AbB is a human monovalent monoclonal antibody against AbA, a human monoclonal antibody (IgG4 subclass) specific for NPR1.

b. Methods

The assays described herein are intended for the quantitative determination of AbB in a sample by acidifying the sample to a pH sufficient to dissociate AbB from its complexes in the sample. Capture and detection reagents specific for AbB are used in the methods and kits to detect and determine the amount or concentration of AbB in the sample.

The methods and kits described herein also provide capture reagent and detection reagent combination for AbB detection and quantification, wherein after the capture reagent binds to AbB, the binding site and the epitope of the detection reagent are available for the detection reagent to bind to AbB.

The capture reagent can be an anti-AbB antibody or antigen-binding fragment thereof. The detection reagent can be an anti-AbB antibody or antigen-binding fragment thereof.

The capture agent can be a mouse anti-AbB antibody (anti-AbB-Ab1 or anti-AbB-Ab2). The detection reagent can be a mouse anti-AbB antibody (anti-AbB-Ab1 or anti-AbB-Ab2).

An interfering agent may be present in the assay, for example, AbA.

The sample acidification can recover at least 70%-100% signal of the analyte, i.e., has a recovery rate of at least about 50%, 55%, 60%, 65%, 70%, 75%, 80%, 85%, 90%, 95% or 100%.

The sample can be acidified to a pH of about 4.0-5.0, for example, a pH of about 4.0, 4.1, 4.2, 4.3, 4.4, 4.5, 4.6, 4.7, 4.8, 4.9 or 5.0.

The sample acidification can be accomplished by the addition of an assay dilution buffer comprising 30 mM acetic acid.

In some exemplary and non-limiting assays, AbB in a biological sample, such as human serum, is quantified by using a mouse anti-AbB antibody (anti-AbB-Ab2) as a capture agent and a different biotinylated mouse anti-AbB antibody (anti-AbB-Ab1) as a detection agent. The amount of AbB is proportional to the signal intensity detected by the detection agent.

The inventions also provide methods for evaluating the effect of sample acidification on assay sensitivity in AbB quantification. The method comprises incubating in AbB-containing sample with neutral or acidic assay dilution buffer (ADB) to acidify the sample, for example, lower the pH of the sample to 5.0-4.0, detecting AbB using the methods and assays of the inventions as described in this disclosure, and comparing the detected value of in AbB in an acidified sample to the value in a neutral sample (for example, pH of 7.4).

The inventions also provide methods for assessing in AbB signal recovery rate in the presence of an interfering agent, for example, AbA. Samples comprising only AbB or AbB and one or more interfering agents (for example, AbA) are diluted with a neutral or an acidic assay dilution buffer (ADB) to acidify the sample, for example, lower the pH of the sample to 5.0-4.0. AbB signals detected from the samples comprising the interfering agent are compared to signals detected from the samples that do not comprise the interfering agent to determine the analyte recovery rate.

The recovery rate of 0.078 ng/mL AbB in the presence of varying amounts of AbAis can be at least 55-100%, for example, at least 50%, 55%, 60%, 65%, 70%, 75%, 80%, 85%, 90%, 95% or 100%.

### G. Methods and Assays for Detection and Quantification of Factor XI

a. Factor XI

Factor XI or FXI is a 160-kDa disulfide-linked dimer of identical 607 amino acid subunits, each containing four 90- or 91-amino acid repeats and a C-terminal trypsin-like catalytic domain. FXI encodes coagulation factor XI that triggers the middle phase of the intrinsic pathway of blood coagulation cascade by activating factor IX protein. Factor XI deficiency, also known as, hemophilia C, plasma thromboplastin antecedent deficiency, or Rosenthal syndrome, is a disorder that can cause abnormal bleeding, especially after trauma or surgery. FXI deficiency is an autosomal recessive disorder that commonly occurs in patients of Ashkenazi Jewish descent.

b. Methods

The assays described herein are intended for the quantitative determination of total Factor XI antigen in a sample, for example, natural and/or recombinant Factor XI, by acidifying the sample to a pH sufficient to dissociate FXI from its complexes in the sample. Capture and detection reagents specific for FXI are used in the methods and kits to detect and determine the amount or concentration of FXI in the sample.

The methods and kits described herein also provide capture reagent and detection reagent combinations for FXI detection and quantification, wherein after the capture reagent binds to FXI, the binding site and the epitope of the detection reagent are available for the detection reagent to bind to FXI.

The capture reagent can be an anti-FXI antibody or antigen-binding fragment thereof. The detection reagent can be an anti-FXI antibody or antigen-binding fragment thereof.

Anti-FXI antibodies as well as methods of making anti-FXI antibodies are known in the art. Anti-FXI antibodies are also commercially available (for example, Affinity Biologicals, Cat #FXI-AG).

An interfering agent may be present in the assay, for example, a human anti-EGFR monoclonal antibody (for example, Int-FXI-Ab1).

The sample acidification can recover at least 70%-100% signal of the FXI, i.e., has a recovery rate of at least 50%, 55%, 60%, 65%, 70%, 75%, 80%, 85%, 90%, 95%, or 100%.

The sample can be acidified to a pH of about 2.0-5.0, for example, a pH of about 2.0. 2.2, 2.4, 2.6, 2.8, 3.0, 3.2, 3.4, 3.6, 3.8, 4.0, 4.1, 4.2, 4.3, 4.4, 4.5. 4.6, 4.7, 4.8, 4.9, or 5.0.

The sample acidification can be accomplished by the addition of an assay dilution buffer comprising 30-90 mM acetic acid, for example, 90 mM, 60 mM, 40 mM, or 30 mM acetic acid.

In some exemplary and non-limiting assays, FXI is detected and quantified in a sample including serum, plasma, saliva, CSF, and urine as described herein. The assay procedures employ a microplate coated with a goat anti-human FXI polyclonal antibody and utilized recombinant monkey FXI as a standard. FXI captured on the plate is detected with goat anti-human FXI polyclonal antibody conjugated to peroxidase (HPA). A peroxidase-specific substrate is then added to achieve a signal intensity that is proportional to the concentration of total FXI.

The inventions also provide methods for evaluating the effect of sample acidification on assay sensitivity in FXI quantification. The methods comprise incubating the FXI-containing sample with neutral or acidic assay dilution buffer (ADB) to acidify the sample, for example, lower the pH of the sample to 5.0-2.0, detecting FXI using the methods and assays of the inventions as described in this disclosure, and comparing the detected value of in FXI in an acidified sample to the value in a neutral sample (for example, pH of 7.4).

The inventions also provide methods for assessing FXI signal recovery rate in the presence of an interfering agent, for example, an anti-FXI antibody, for example, Int-FXI-Ab1. Samples comprising FXI only or FXI with one or more interfering agents (for example, Int-FXI-Ab1) are diluted with a neutral or acidic assay dilution buffer (ADB) to acidify the sample, for example, lower the pH of the sample to 5.0-2.0. FXI signals detected from the samples comprising the interfering agent are compared to signals detected from the samples that do not comprise the interfering agent to determine the analyte recovery rate.

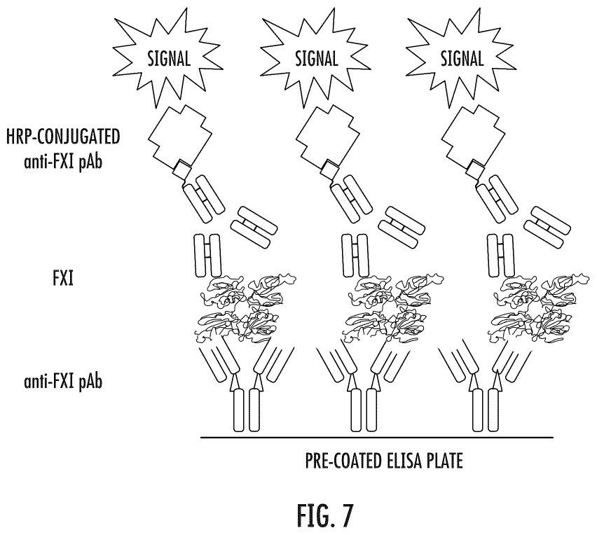

A schematic of an exemplary assay approach for determining concentration of factor XI (FXI) in a sample is depicted in FIG. 7. An anti-FXI antibody is bound to a solid plate to capture FXI. An HRP-conjugated anti-factor XI antibody is used to detect the captured FXI.

TABLE A
List of analytes, capture reagents, detection reagents,
and interfering agents used in certain examples

| Name | Descriptions and uses in some examples |
|---|---|
| AbA | A human IgG4 monoclonal antibody against NPR1 |
| | Analyte |
| | Interfering agent for AbB |
| Anti-AbA-Ab1 | A mouse anti-AbA monoclonal antibody |
| | Capture reagent |
| Anti-AbA-Ab2 | A second mouse anti-AbA monoclonal antibody |
| | Detection reagent |
| Anti-hIgG4Fc | A mouse anti-human IgG4 Fc specific monoclonal |
| | antibody |
| | Detection reagent |
| Int-AbA-Ab1 | A human monoclonal antibody against AbA |
| | Intefering agent |
| Int-AbA-Ab2 | A second human monoclonal antibody against AbA |
| | Intefering agent |
| Int-AbA-Ab3 | A third human monovalent monoclonal antibody against |
| | AbA |
| | Intefering agent |
| AbB | A human monovalent monoclonal antibody against AbA |
| | Analyte |
| | Intefering agent for AbA |
| Anti-AbB-Ab1 | A mouse anti-AbB antibody |
| Anti-AbB-Ab2 | A second mouse anti-AbB antibody |
| Anti-IL2Rγ- | A human anti-IL2Rγ gamma monoclonal antibody |
| Ab1 | Capture or detection reagent for IL2Rγ |
| Anti-IL2Rγ- | A second human anti-IL2Rγ gamma monoclonal |
| Ab2 | antibody |
| | Capture or detection reagent for IL2Rγ |
| Int-IL2Rγ-Ab1 | A human anti-IL2Rγ gamma monoclonal antibody |
| | Interfering agent for IL2Rγ |
| Int-EGFR-Ab1 | A bispecific human monoclonal antibody against EGFR |
| | Intefering reagent |
| Int-FXI-Ab1 | An anti-factor XI antibody |
| | Interfering agent |

### VII. EXAMPLES

The inventions are further described by the following Examples, which do not limit the inventions in any manner. The order of performance of the below examples can be altered or combined as determined by the person of skill in the art in view of the teachings and data contained herein.

### Example 1: Assays for Detecting IL2Rγ

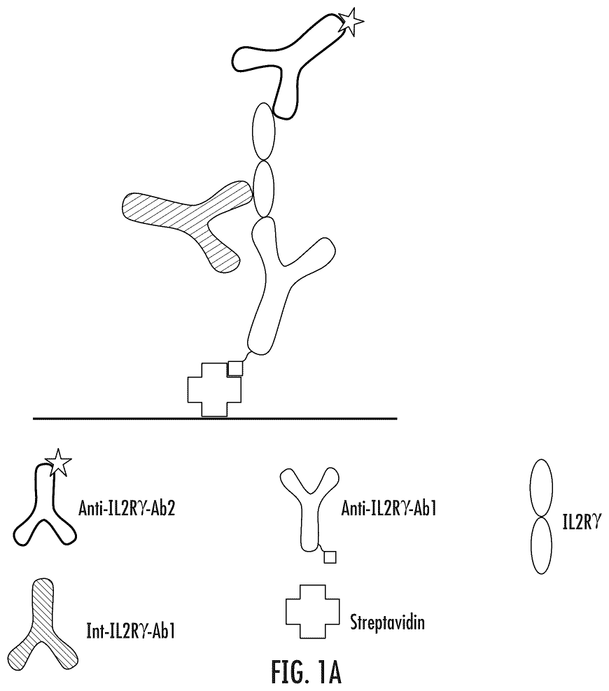

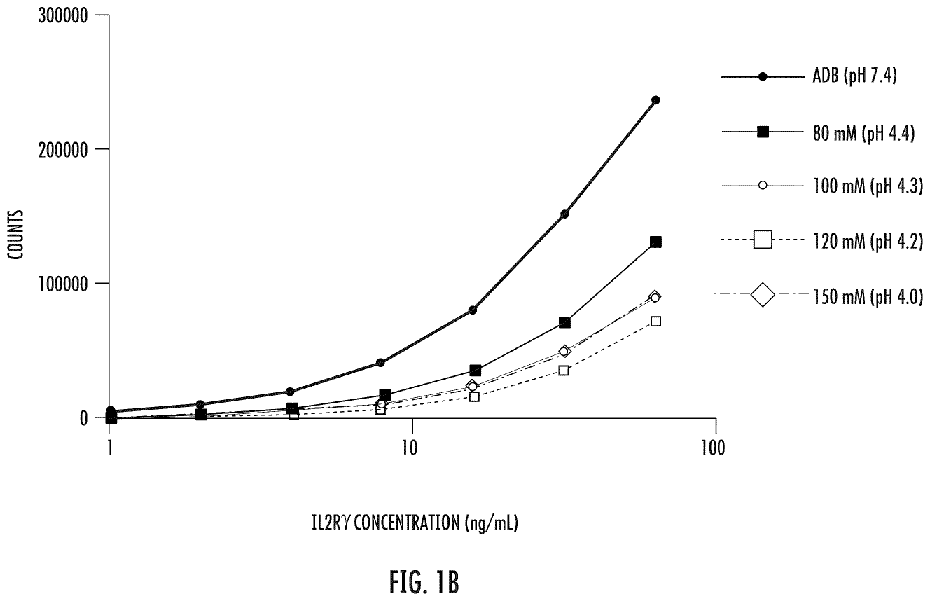

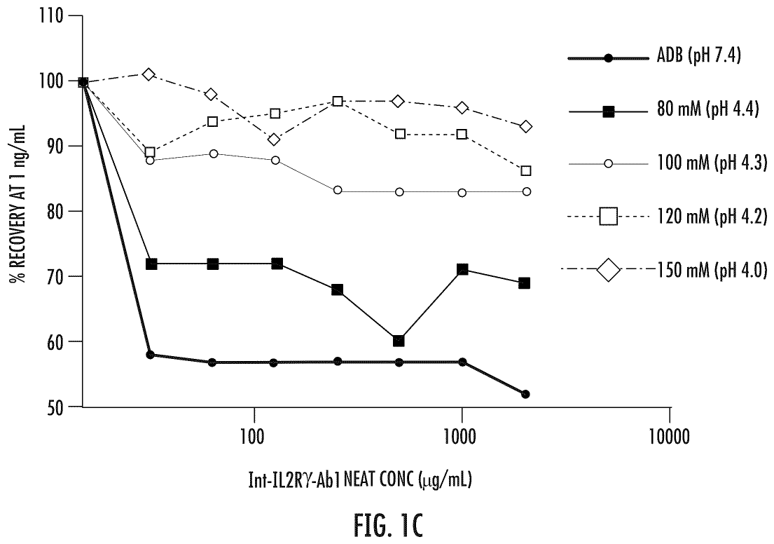

This study was carried out to improve assay sensitivity for IL2Rγ detection and quantification by optimizing the procedures and pH conditions that minimize potential drug interference in samples. This study also examined whether neutralization of the acidified sample is necessary for the capture reagent to capture IL2Rγ in the sample. Briefly, a biotinylated human anti-IL2Rγ antibody anti-IL2Rγ-Ab1 was bound to a solid support spotted with streptavidin to capture IL2Rγ, and a ruthenium-labeled human anti-IL2Rγ antibody anti-IL2Rγ-Ab2 was used to detect the captured human IL2Rγ. Signal intensity from the ruthenium-label that is proportional to the concentration of total IL2Rγ in a sample (FIG. 1A) is generated using MSD Read Buffer. An interfering agent, for example, human anti-IL2Rγ antibody, Int-IL2Rγ-Ab1, may be present in the samples.

Materials, Methods and Results

A streptavidin-coated microplate was blocked by adding 300 μL/well of 5% BSA blocking buffer and incubated for 1 to 4 hours at room temperature. A biotinylated anti-IL2Rγ antibody (Bio-anti-IL2Rγ-Ab1) at the concentration of 2 μg/mL was added to the microplate and incubated for about 1 hour. Samples were diluted at a 1:4 ratio in a neutral assay dilution buffer (ADB) or ADB containing 80 mM to 150 mM acetic acid, wherein both buffers contained 5% bovine serum albumin (BSA). Without neutralization, 50 μL diluted samples were incubated in the antibody-coated microplate for 1 hour. After the removal of the diluted samples from each well, 50 μL ruthenium-labeled anti-IL2Rγ antibody (Ru-anti-IL2Rγ-Ab2) at the concentration of 200 ng/ml was added to the microplate and incubated for about 1 hour. At the end of the experiment, 150 μL/well of 2×MSD GOLD read buffer was added to the assay plate. Data was obtained on an MSD plate reader at OD450-540 nm within 10 minutes after addition of the read buffer.

Data Analysis

Signal count generated by the ruthenium label and read by the MSD reader of each sample was plotted against a fitted calibrator curve using 4PLV equation based on the nominal concentration of each calibrator. Concentration of IL2Rγ was extrapolated based on the count values corresponding to known concentrations of IL2Rγ standards.

Results

The overall signal reduced when the samples were diluted with ADB containing 80-150 mM acetic acid, which decreased the pH of the samples from 7.4 to the range of 4.4-4.0 (see FIG. 1B). The decrease in signal leads to a higher lower limit of quantitation (LLOQ), which is about 5-10 fold greater than the baseline samples at neutral pH. In other words, the sensitivity of the assay was lowered when the samples were acidified.

In the presence of interfering agent Int-IL2Rγ-Ab1 (Int-IL2Rγ-Ab1), signal from IL2Rγ reduced below 60% in the neutral assay dilution buffer. Decreasing the pH of the sample by adding ADB of decreasing pH increased signal recovery, and a pH around 4.2 resulted in an acceptable tolerance to interfering agent (see FIG. 1C).

Surprisingly, while acidification led to dissociation of Int-IL2Rγ-Ab1 from IL2Rγ and reduced interference from Int-IL2Rγ-Ab1, it did not affect Bio-anti-IL2Rγ-Ab1 and IL2Rγ binding efficiency at a pH as low as 4.0, which is evidenced by the signal recovery of up to 90-100%. This suggests that neutralization of the acidified sample prior to incubation of the sample with the capture-agent is not required, thus simplifying the assay.

### Example 2: Assays for Detecting EGFR

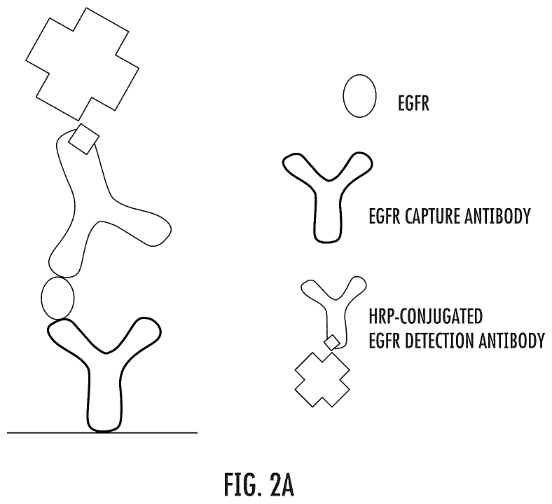

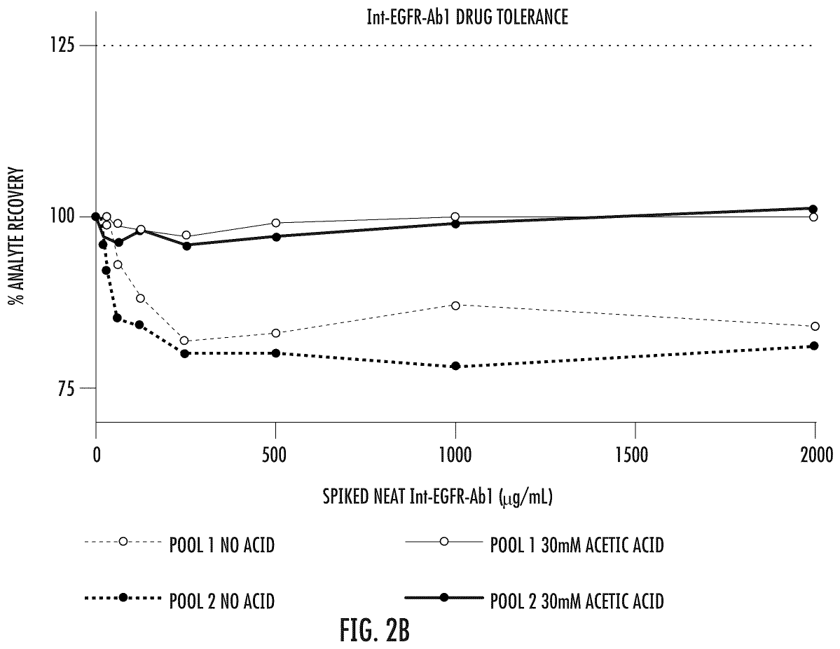

In an exemplary assay for determining the concentration of total soluble EGFR in a sample, a EGFR capture antibody was pre-coated to a solid support to capture the soluble EGFR in a samples. A HRP-conjugted EGFR detection antibody was used to detect the captured EGFR (FIG. 2A).

Materials, Methods and Results

Preparation of 50×EGFR reference standards: Prior to the experiment, 50×EGFR standards used for standard curve were prepared in calibrator diluent by serially diluting 1000 ng/ml EGFR solution at 1:2 dilution to obtain standards containing 1000, 500, 250, 125, 62.5, 31.3, 15.6, and 0 ng/mL EGFR.

Preparation of 50×EGFR quality control: Samples with a known concentration of EGFR were used as quality controls to verify the assay's performance. 50× high quality control (HQC), mid-quality control (MQC), low quality control (LQC) were prepared in calibrator diluent at the concentration of 750 ng/mL, 120 ng/mL, and 45 ng/mL.

Preparation of standards, Quality controls and Samples in assay buffer: Each of the 50 standards, 50×QC and sample was diluted 1:50 in calibrator diluent with 20 mM acetic acid.

Assay plate preparation: The microplates coated with an anti-EGFR polyclonal antibody and the Detection Antibody are part of the Human EGFR Quantikine ELISA Kit (R&D Systems Quantikine Kit Cat #DEGFR0). Following an addition of 100 μl/well acidic assay diluent to the anti-EGFR microplate coated with anti-EGFR antibody (R&D Systems, Cat/Part #893730), another 50 μl/well of calibrators, QCs, and study acidified samples (without neutralization) was added to the microplate in duplicate. The plate was covered and incubated for 120±10 minutes at room temperature, shaking at 200 rpm. After a 5× wash with 300 μl/well of 1× wash buffer, the detection antibody (horseradish Peroxidase-conjugated anti-EGFR polyclonal antibody, R&D Systems, Cat/Part #893731) was add to the microplate at 200 μL/well. The plate was covered and incubated for another 120±10 minutes at room temperature in the dark, shaking at 200 rpm. At the end of the experiment, 50 μl/well of stop solution was added to the microplate, and absorbance was measured using a microplate reader at OD450-540 nm within 30 minutes of Stop Solution addition.

Data Analysis

Means optical density (OD) of each sample was plot against the fitted calibrator curve using 4PLV equation based on the nominal concentration of each calibrator. Concentration of EGFR was extrapolated based on the OD values corresponding to known concentrations of EGFR standards.

Results

The effect of sample acidification on Int-EGFR-Ab1 drug tolerance was evaluated using the methods disclosed herein. More specifically, 0 to 2 mg/mL interfering agent Int-EGFR-Ab1 was spiked into EGFR-containing samples to test the effect of pH on Int-EGFR-Ab1:EGFR dissociation. Samples were diluted 1:50 in RD5L assay buffer with or without 30 mM acetic acid. 100 μl of RD1-72 kit assay buffer diluent was added to each well of the coated-microplate, and 50 μl of the diluted samples was mixed in each well with the RD1-72 diluent. The diluted samples were incubated in the wells for about 2 hours, followed by an incubation of the HRP-conjugated anti-EGFR antibody for about 2 hours. After 5× wash with the wash buffer, 200 μl of TMB substrate was added to each well and incubated in the dark for about 25 minutes, and 50 μl of a stop solution (2N sulfuric acid) was added to stop the color development. Absorbance was measured at OD₄₅₀₋₅₄₀ using a microplate reader (see FIG. 2A for a schematic of the assay).

The results according to FIG. 2B demonstrated that detection of EGFR was inhibited in the neutral ADB (pH=7.4), while sample acidification significantly increased dissociation of EGFR complexes and detection of EGFR. Furthermore, assay dilution buffer with 30 mM acetic acid (pH=4.5) significantly improved the EGFR assay tolerance to Int-EGFR-Ab1; specifically, and the analyte recovery rate was about 100% in the presence of Int-EGFR-Ab1 of up to 2 mg/mL.

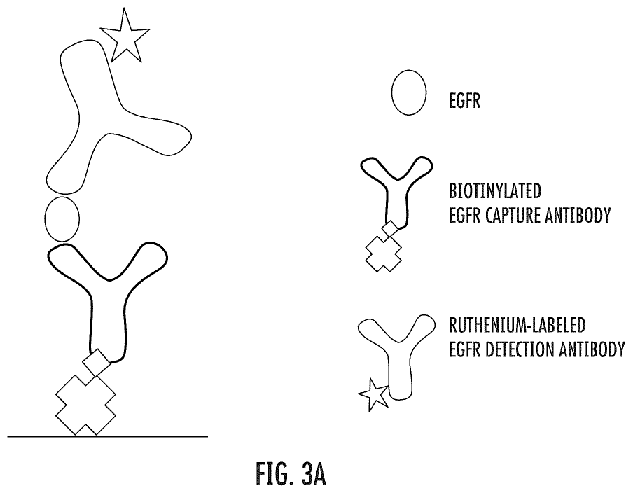

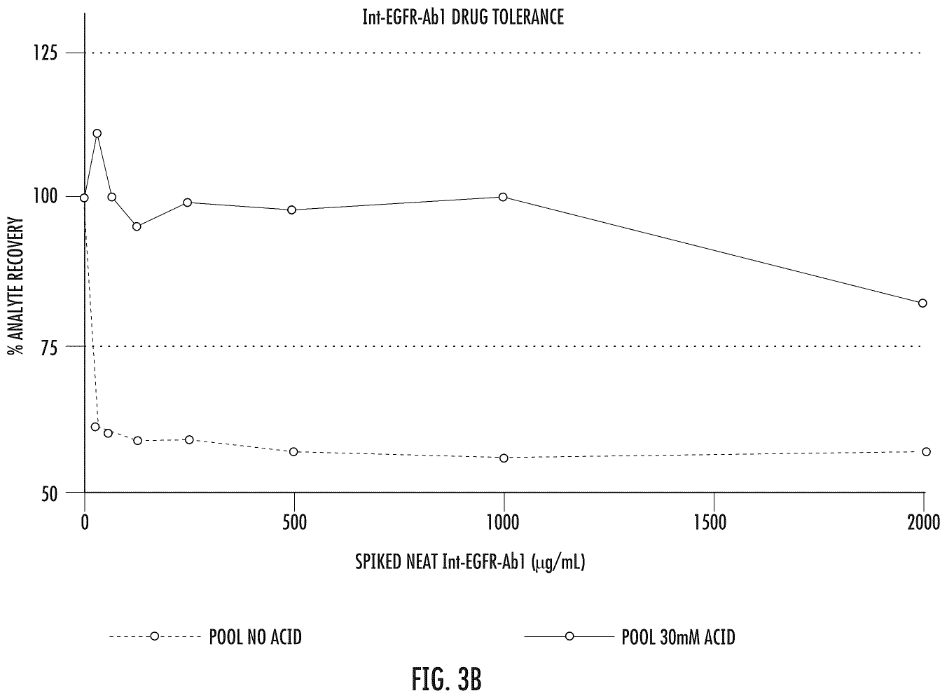

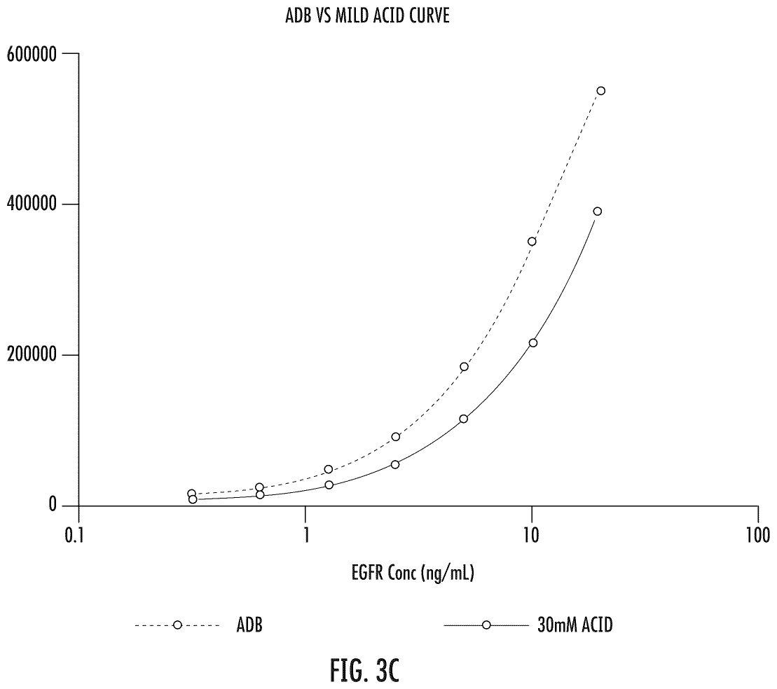

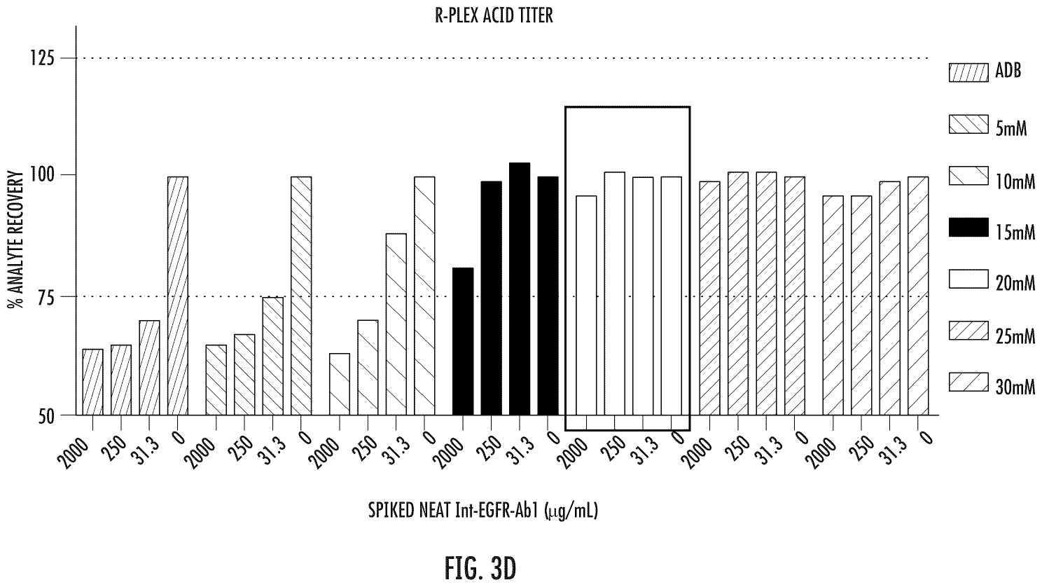

Similar improvement in analyte detection was observed in a similar assay, which used a biotinylated EGFR antibody as capture reagent and a Ruthenium-labeled EGFR antibody as detection reagent (FIG. 3A). These antibodies are from the MSD R-PLEX Human EGFR antibody Set (Cat #F21 N5), and the capture and detection are both goat polyclonal antibodies. After sample acidification, analyte recovery rate was about 100% at a concentration of Int-EGFR-Ab1 of up to 1 mg/mL, and analyte recover rate was above 75% in the presence of 2 mg/ML of Int-EGFR-Ab1 (FIG. 3B).

In the mildly acidic samples, detection of EGFR was slightly lower than in neutral ADB buffer (FIG. 3C), which suggests that EGFR and anti-EGFR antibody binding according to the assay in FIG. 3A is somewhat pH sensitive, and acidic ADB slightly decreased the detection of EGFR compared to the samples treated with neutral ADB.

In FIG. 3D, the effects of sample acidification to various pH on EGFR recovery rate was evaluated in EGFR-containing sample spiked with 0 μg/mL, 31.3 μg/mL, 250 μg/mL, and 2000 μg/mL neat Int-EGFR-Ab1 using a R-PLEX Acid Titer assay. The data indicates that lowering the sample pH increased tolerance of EGFR to interfering agent Int-EGFR-Ab1. Noticeably, with sample dilution buffers containing 5-15 mM acetic acid, the EGFR recovery rate was negatively affected by high concentration of Int-EGFR-Ab1 (2000 μg/mL), whereas, ADB with 20-30 mM acetic acid (sample pH of 5-4.5) stabilized the tolerance of EGFR to up to 2 mg/mL of Int-EGFR-Ab1 and had more than 90% recovery rate. The overall data also showed that the assay very effectively recovered signals from EGFR in the acidified sample even when the the acidified samples were not neutralized prior to the binding step with the capture antibody.

The assay was tested with samples containing different amount of serum to determine its lower limit of detection or lower limit of quantitation. At a pH of about 5.0, in the presence of 2% human serum, EGFR at as low as 0.31 ng/mL was detectable. In the presence of undiluted human serum, EGFR at as low as 15.6 ng/mL was detectable.

### Example 3: Immunoassays for Detecting AbA

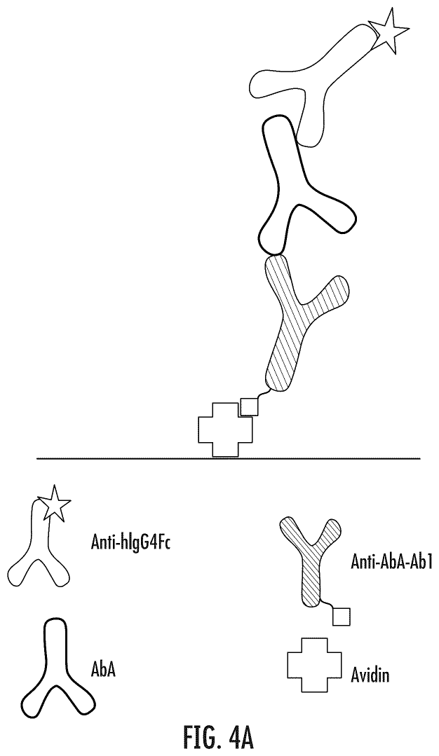

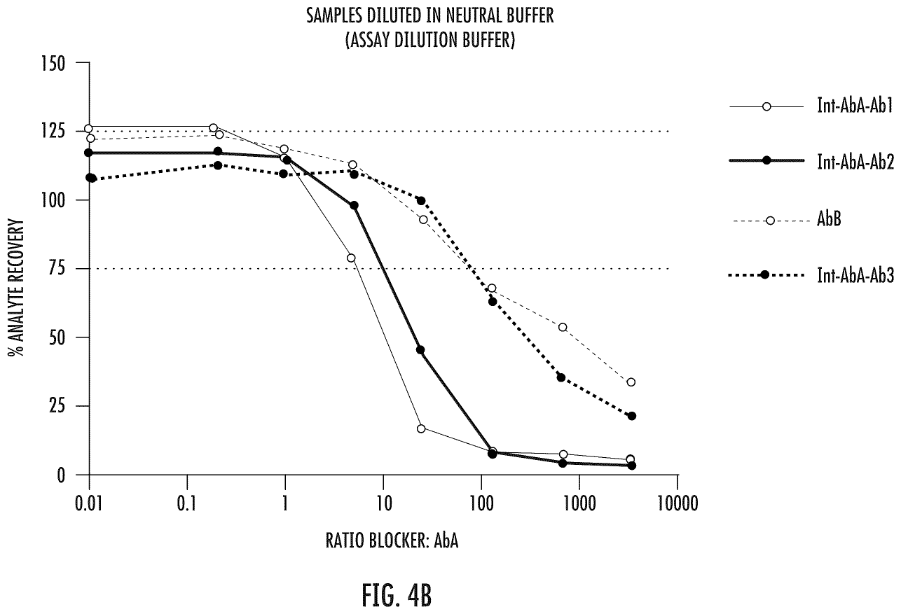

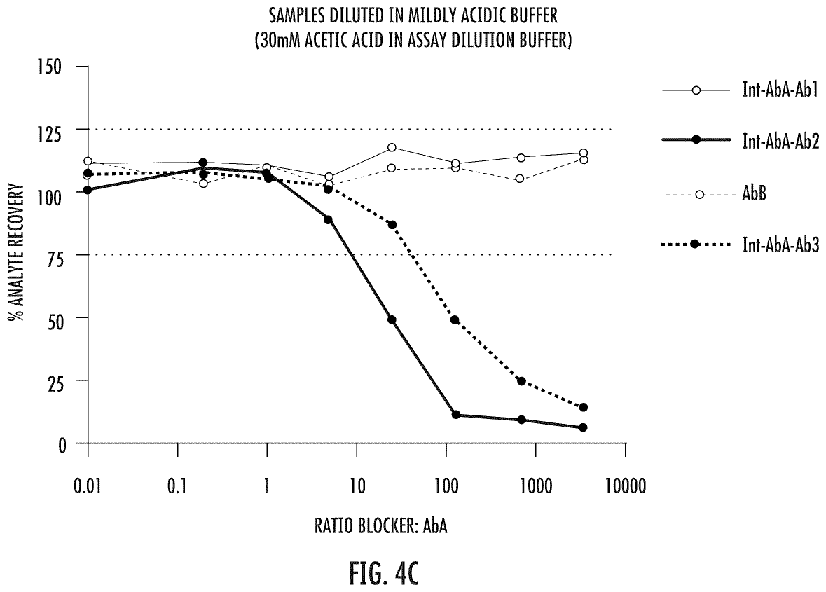

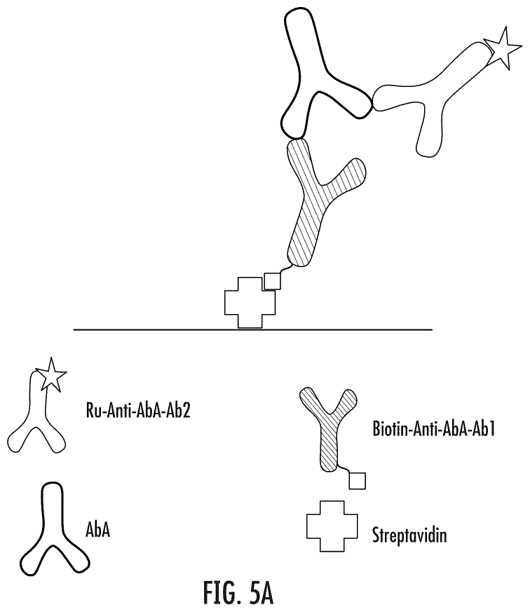

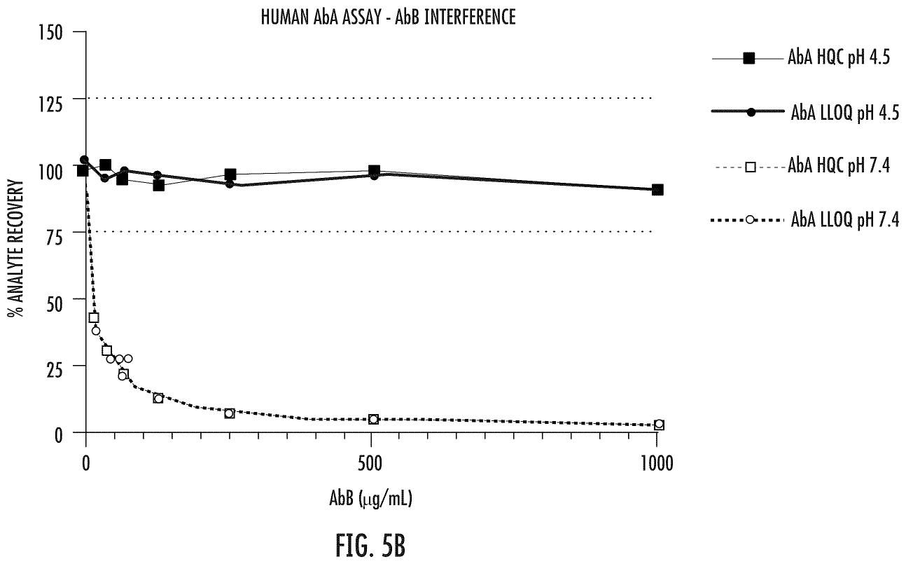

In an exemplary assay for determining the concentration of AbA, a human IgG4 monoclonal antibody against NPR1, in a sample, a biotinylated mouse anti-AbA monoclonal antibody (anti-AbA-Ab1) is immobilized to avidin-coated plate as a capture agent, and a ruthenium-conjugated mouse anti-human IgG4 Fc antibody (anti-hIgG4Fc) was used to detect the captured AbA. An electrochemiluminescent signal is then generated by the ruthenium label when voltage is applied to the plate by the MSD reader (FIG. 4A). In another exemplary assay, ruthenylated anti-AbA-Ab2, a mouse anti-AbA monoclonal antibody, was used as the detection agent. (FIG. 5A).

Materials and Methods

Preparation of 50× AbA standards: Prior to the experiment, 50× AbA standards used for standard curve were prepared in human serum by serially diluting 1 mg/ml of AbA placebo at 1:3 dilution to obtain standards containing 10000, 3333, 1111, 370, 123, 41.2, 13.7 and 0 ng/mL AbA. Frozen standards may be used up to 90 days after the storage date.

Preparation of 50× AbA quality control: Samples with a known concentration of AbA were used as quality controls to verify the assay's performance. 50× high quality control (HQC), mid quality control (MQC), low quality control (LQC) were prepared in human serum at the concentration of 7500 ng/mL, 400 ng/mL, and 40 ng/mL.

Preparation of standards, Quality controls and Samples in assay buffer: 30 mM acidic assay dilution buffer (ADB) was prepared by diluting 300 mM acetic acid with ADB, which was then used to prepare Assay buffer that contains 10 μg/mL mouse IgG. Each 50 standards, 50×QC and sample was diluted 1:50 in assay buffer for assay procedure.

Assay plate preparation: A streptavidin-coated microplate was blocked by adding 300 μL/well of 5% BSA blocking buffer and incubated for 1 to 2 hours at room temperature if used in the same day. The blocked microplate was washed 3× with 300 μL/well of 1× washing buffer using the MSD_PLATE_3× wash program. Capture antibody (Biotin-anti-AbA-Ab1) was prepared at 1 μg/mL in ADB and then added to the microplate at 50 μL/well. The microplate was then covered and incubated for 1 to 2 hours at room temperature, shaking at 400 pm during the incubation. The microplate was then washed 3× with 300 μL/well of 1× wash buffer using the MSD_PLATE_3× wash program.

Assay procedure: The prepared Standards, QCs, and Samples (unneutralized) were added 50 μL/well to the microplate in duplicate, which was covered and incubated for 60±10 minutes at room temperature, shaking at 400 rpm during the incubation. The microplate was then washed 3× with 300 μL/well of 1× wash buffer using the MSD_PLATE_3× wash program. The detection antibody (Ru-anti-AbA-Ab2) was prepared at 400 ng/mL in ADB and added to the plate at 50 μL/well for a 60±10 minutes incubation, shaking at 400 rpm. 150 μL/well of MSD GOLD Read buffer (MSD, Cat #R92TG) was added to the assay plate following a 3× wash with 300 uL/well of 1× Wash Buffer using the MSD_PLATE_3× wash program. Data was obtained on an MSD plate reader within 10 minutes after addition of the Read buffer.

Data Analysis

Mean Counts (Signal) of each sample was plot against the fitted calibrator curve using 4PLV equation based on the nominal concentration of each calibrator. Concentration of AbA was extrapolated based on the Count values corresponding to known concentrations of AbA standards.

Results

The assay depicted in FIGS. 5A-5B employed a biotinylated anti-AbA-Ab1 as a capture agent, and ruthenium-labeled anti-AbA-Ab2 as a detection agent. Both anti-AbA-Ab1 and anti-AbA-Ab2 bind to the CDR region of the AbA (FIG. 5A). The tolerance of different levels of AbA to interfering agent AbB was evaluated under both neutral (pH=7.4) and acidic (pH=4.5) ADB. Namely, the high quality control sample (HQC) contains 7.5 μg/mL AbA, and the lower limit of quantitation sample (LLOQ) contains 13.7 ng/mL. Each sample was diluted 1:50 in either neutral buffer (pH 7.4) containing 10 μg/mL mouse IgG or mild acidic buffer containing 30 mM acetic acid and 10 μg/mL mouse IgG. AbA recovery rate was measured in the presence of interfering agent AbB of up to 1 mg/mL.

As a result, both HQC and LLOQ samples had a similar AbA recovery rate that is above 95% in the presence of interfering agent AbB (FIG. 5B). Furthermore, mild sample acidification effectively dissociated AbB:AbA complex, resulting in an acceptable tolerance to at least 1 mg/mL AbB at the pH 4.5.

In a similar assay, ruthenylated anti-hIgG4Fc was used as the detection agent. (FIG. 4A). To evaluate the effect of sample acidification on AbA recovery rate and assay sensitivity, a blocker selecting from one of the interfering agents Int-AbA-Ab1, Int-AbA-Ab2, AbB and Int-AbA-Ab3 (AbA reversal agents that are human anti-AbA monoclonal antibodies) was added to the AbA-containing samples to reach a blocker:AbA ratio of up to ˜3300:1. AbA recovery rate was evaluated by a 1:50 sample dilution in either a neutral ADB containing 10 μg/mL mouse IgG (pH=7.4) (FIG. 4B) or an acidic ADB containing 30 mM acetic acid with 10 μg/mL mouse IgG (pH=˜4.5) (FIG. 4C). The sandwich immunoassay was carried out in steps similar to what described above.

The results according to FIG. 4B suggest that after dilution in a neutral buffer, the presence of interfering agents Int-AbA-Ab1, Int-AbA-Ab2, AbB and Int-AbA-Ab3 inhibited detection of AbA. This was reflected by a decrease of AbA recovery rate when the interfering agent:AbA ratio (blocker:AbA ratio) was greater than 10:1. More specifically, AbA recovery rate dropped below 75% when the ratio of Int-AbA-Ab1:AbA and Int-AbA-Ab2:AbA was greater than 10:1, and the ratio of AbB:AbA and Int-AbA-Ab3:AbA was greater than 100:1. When the ratio of Int-AbA-Ab1:AbA and Int-AbA-Ab2:AbA was 100:1 and above, AbA recovery rate was reduced to less than 10%.

FIG. 4C demonstrated that sample acidification effectively prevented the interference of Int-AbA-Ab1 and AbB, but not Int-AbA-Ab2 and Int-AbA-Ab3, in AbA detection when the blocker:AbA ratio was greater than 10:1. In the assay dilution buffer that contains 30 mM acetic acid, AbA signal was completely recovered when the Int-AbA-Ab1:AbA and AbB:AbA ratio was up to ˜3300:1. However, the recovery rate of AbA remained as low as 6% for Int-AbA-Ab2 and 14% for Int-AbA-Ab3 when the blocker:AbA ratio was ˜3300:1.

These data suggest that sample acidification selectively increased the detection of AbA in the presence of interfering agents.

Data presented in FIGS. 4B, 4C, and 5B demonstrated that sample acidification in the immunoassay helped prevent drug interference caused by Int-AbA-Ab1 and AbB without affecting AbA quantification sensitivity. The overall data also showed that the assay very effectively recovered signals from AbA in the acidified sample even when the the acidified samples were not neutralized prior to the binding step with the capture antibody.

The assay was tested with samples containing different amount of serum to determine its lower limit of detection or lower limit of quantitation. At a pH of about 4.5, in the presence of 2% human serum, AbA at as low as 0.27 ng/mL was detectable. In the presence of undiluted human serum, AbA at as low as 13.7 ng/mL was detectable.

### Example 4: Assays for Detecting a Reversal Agent of Human Monoclonal IgG4 Antibody Against NPR1 (AbB)

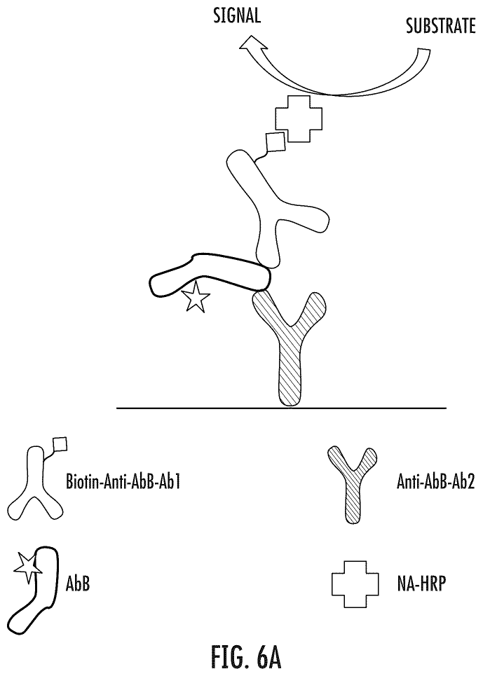

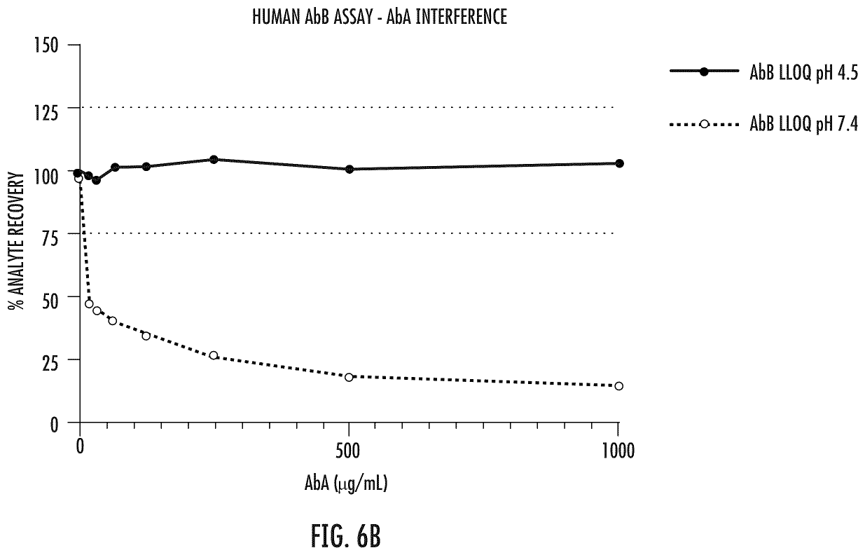

This experiment relates to an assay similar to those described above in determining the concentrations of total AbB in a sample. In one exemplary assay, an anti-AbB antibody was immobilized to solid plate as a capture agent, and a different biotinylated mouse anti-AbB antibody (biotin-anti-AbB-Ab1) was used to detect captured AbB. NeutrAvidin-HRP (ThermoFisher cat #31001) and Pico chemiluminescence substrate are used to produce read-out for the amount of the detection reagent bound to the captured AbB and for the amount and concentration of AbB (FIG. 6A). The samples may contain an interfering reagent, AbA.

Results

To evaluate sample acidification on the tolerance of AbB to interfering agent AbA, the recovery rate of 0.078 μg/mL (LLOQ) AbB was measured in the presence of 31.25 μg/mL to 2 mg/mL AbA at pH=7.4 and pH=4.5 (FIG. 6B). Compared to neutral assay binding buffer (ADB), acidic ADB dramatically enhanced the recovery rate to 97-105%, demonstrating the effectiveness of sample acidification in improving sensitivity of AbB quantification assay and its tolerance to AbA (see FIG. 6B). The data also showed that the assay very effectively recovered signals from AbB in the acidified sample even when the the acidified samples were not neutralized prior to the binding step with the capture antibody.

1. A method for detecting an analyte in a sample comprising the steps of:
(i) diluting a sample comprising the analyte with a weak acid to produce an acidified sample;
(ii) adding the acidified sample directly to a solid support without neutralizing the acidified sample, wherein the solid support is coated with a capture reagent that can bind to the analyte;
(iii) removing the acidified sample from the solid support after a first period of incubation;
(iv) adding a detection reagent directly to the solid support;
(v) removing the detection reagent from the solid support after a second period of incubation; and
(vi) detecting the detection reagent bound to the analyte captured by the capture reagent, wherein the quantity of the detection reagent detected correlates to the quantity of the analyte in the sample.

2.-3. (canceled)

4. The method according to claim 1, wherein the acidified sample has a pH of about 3.0 to 6.5, about 4.1, about 4.4, or about 4.5.

5. (canceled)

6. The method according to claim 1, wherein the analyte is IL2Rγ, EGFR, an antibody specific for natriuretic peptide receptor 1, a human monovalent monoclonal antibody against the anti-NPR1 antibody, or factor XI.

7. The method according to claim 1, wherein the sample is a biological fluid selected from the group consisting of blood, serum, plasma, cerebrospinal fluid (CSF), urine, and saliva.

8. The method according to a claim 1, wherein the sample is from (a) a subject having a disease or disorder, (b) a subject suspected of having a disease or disorder, or (c) a subject who was administered a substance and/or a drug product.

9.-11. (canceled)

12. The method according to claim 1, wherein the weak acid is an acid that does not completely dissociate into its ions in an aqueous solution selected from the group consisting of acetic acid, citric acid, formic acid, lactic acid, phosphoric acid, and PIPES.

13. (canceled)

14. The method according to claim 1, wherein the capture reagent is an antibody, the detection reagent is an antibody, or both the capture reagent and detection reagent are antibodies.

15. (canceled)

16. The method according to claim 14, wherein the capture reagent binds the analyte with a dissociation constant (K_{D}) value of ≤1 μM, ≤100 nM, ≤50 nM, ≤25 nM, K 20 nM, ≤15 nM, ≤10 nM, ≤5 nM, ≤2 nM, ≤1 nM, ≤0.1 nM, ≤0.01 nM, or ≤0.001 nM.

17. The method according to claim 1, further comprising the step of identifying a capture reagent that can bind to the analyte at the pH of the acidified sample.

18.-21. (canceled)

22. The method according to claim 1, wherein the detection reagent is conjugated to a detectable label.

23.-26. (canceled)

27. The method according to claim 1, wherein the sample comprises an interfering agent that binds to the analyte.

28. The method according to claim 27, further comprising the step of determining a working pH for the acidified sample at which the interfering agent dissociates partially or completely from the analyte.

29. The method according to claim 27, wherein the method recovers at least 50%, 55%, 60%, 65%, 70%, 75%, 80%, 85%, 90%, 95%, 100% analyte signal in a sample comprising the interfering agent as compared to a sample that does not comprise the interfering agent.

30. (canceled)

31. The method according to claim 1, wherein the quantity of the analyte is determined by correlating the amount of detected detection reagent to a predetermined reference standard.

32. A kit for use in the method according to claim 1, wherein the kit comprises the capture reagent, the detection reagent, and a dilution buffer comprising the weak acid.

33. A method for detecting an analyte in a sample comprising the steps of:
(i) diluting a sample comprising the analyte with a weak acid to produce an acidified sample;
(ii) adding a capture reagent to the acidified sample without neutralizing the acidified sample, wherein the capture reagent specifically binds to the analyte,
(iii) adding the mixture comprising the acidified sample and the capture reagent to a solid support, wherein the solid support binds specifically to the capture reagent;
(iv) removing the mixture of acidified sample and capture reagent from the solid support after a first period of incubation;
(vi) adding a detection reagent directly to the solid support;
(vii) removing the detection reagent from the solid support after a second period of incubation; and
(viii) detecting the detection reagent bound to the analyte captured by the capture reagent, wherein the quantity of the detection reagent detected correlates to the quantity of the analyte in the sample.

34.-35. (canceled)

36. The method according to claim 33, wherein the acidified sample has a pH of about 3.0 to 6.5, about 4.1, about 4.4, or about 4.5.

37.-38. (canceled)

39. The method according to claim 33, wherein the sample is a biological fluid selected from the group consisting of blood, serum, plasma, cerebrospinal fluid (CSF), urine, and saliva.

40.-43. (canceled)

44. The method according to claim 33, wherein the weak acid is an acid that does not completely dissociate into its ions in an aqueous solution selected from the group consisting of acetic acid, citric acid, formic acid, lactic acid, phosphoric acid, and PIPES.

45. (canceled)

46. The method according to claim 33, wherein the capture reagent is an antibody, the detection reagent is an antibody, or both the capture reagent and detection reagent are antibodies.

47.-48. (canceled)

49. The method according to claim 33, further comprising the step of identifying a capture reagent that can bind to the analyte at the pH of the acidified sample.

50.-53. (canceled)

54. The method according to claim 33, wherein the detection reagent is conjugated to a detectable label.

55.-58. (canceled)

59. The method according to claim 33, wherein the sample comprises an interfering agent that binds to the analyte.

60. (canceled)

61. The method according to claim 59, wherein the method recovers at least 50%, 55%, 60%, 65%, 70%, 75%, 80%, 85%, 90%, 95% or 100% analyte signal in a sample comprising the interfering agent as compared to a sample that does not comprise the interfering agent.

62.-64. (canceled)
